SEQUENCE LISTING (7 sequences)

SEQ ID NO: 1 - protein, 8 aa, synthetic construct
SIINFEKL

SEQ ID NO: 2 - DNA, 73 bp, synthetic construct
  source 1..73: mol_type = other DNA
gtgaaataccgcacagatgcgtttgtataaatgtttttttcatttatactttaagagcgccacgtagcccagc

SEQ ID NO: 3 - DNA, 92 bp, synthetic construct
  source 1..92: mol_type = other DNA
gtgaaataccgcacagatgcgtttgggttaacatctagattctatttttagaatctagatgttaaccctttaagagcgccacgtagcccagc

SEQ ID NO: 4 - DNA, 92 bp, synthetic construct
  source 1..92: mol_type = other DNA
gtgaaataccgcacagatgcgtttcgccgcgggccggcgcgcggttttccgcgcgccggcccgcggcgtttaagagcgccacgtagcccagc

SEQ ID NO: 5 - DNA, 24 bp, synthetic construct
  source 1..24: mol_type = other DNA
cgcatctgtgcggtatttcacttt

SEQ ID NO: 6 - DNA, 24 bp, synthetic construct
  source 1..24: mol_type = other DNA
tttgctgggctacgtggcgctctt

SEQ ID NO: 7 - DNA, 15 bp, synthetic construct
  source 1..15: mol_type = other DNA
aaaaaacatttatac

(19) United States
(12) Patent Application Publication
(10) Pub. No.: US 2026/0092918 A1
(43) Pub. Date: Apr. 2, 2026

(54) Molecular Tension Probes and Methods to Determine or Monitor the Efficacy of Cancer and Other Immune Therapies

(71) Applicants: Emory University, Atlanta, GA (US); Children's Healthcare of Atlanta, Inc., Atlanta, GA (US)

(72) Inventors: Anna V. Kellner, Atlanta, GA (US); Khalid Salaita, Atlanta, GA (US); Curtis Henry, Atlanta, GA (US)

(21) Appl. No.: 19/112,157

(22) PCT Filed: Sep. 15, 2023
(86) PCT No.: PCT/US2023/074374

§ 371 Date: Mar. 14, 2025

Related U.S. Application Data
(60) Provisional application No. 63/407,211, filed on Sep. 16, 2022.

(51) Int. Cl. G01N 33/569; G01N 21/64; G01N 33/575

(52) U.S. Cl. CPC G01N 33/56972; G01N 21/6428; G01N 33/5759; G01N 2021/6432; G01N 2333/7051

(57) ABSTRACT

This disclosure contemplates methods of detecting, measuring, and/or quantifying a tensional force on cells that express an antigen. In certain embodiments, this disclosure relates to methods and devices disclosed herein used to determine or monitor whether a cancer therapy or other immunotherapy is appropriate for a specific patient. In certain embodiments, the therapy is administering blinatumomab or other bispecific engager to a patient.

### CROSS-REFERENCE TO RELATED APPLICATIONS

This application claims the benefit of U.S. Provisional Application No. 63/407,211 filed Sep. 16, 2022. The entirety of this application is hereby incorporated by reference for all purposes.

### STATEMENT REGARDING FEDERALLY SPONSORED RESEARCH OR DEVELOPMENT

This invention was made with government support under GM124472 and GM131099 awarded by the National Institutes of Health. The government has certain rights in the invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 22192PCT.xml. The XML file is 8 KB, was created on Sep. 14, 2023, and is being submitted electronically via the USPTO patent electronic filing system.

### BACKGROUND

Cancer is a major health problem. The immune system is designed to manage the proliferation of cancerous cells. However, the inability of the immune system to manage uncontrolled growth is common in cancer patients. Many approved and candidate anti-cancer treatments are based on facilitating the activation of immune cells, e.g., T-cells, to engage and eliminate cancerous cells. However, these treatments are not universally effective. Deciding on how to prioritize one cancer therapy over another can affect the ultimate outcome as cancer often becomes progressively more difficult to treat over time. In addition, utilizing immune therapies in different patients can be problematic. Some patients may be non-responsive to a specific therapy while in another patient receiving the same therapy may cause life threatening adverse reactions. Thus, there is a need to identify improved methods for determining likelihood that a particular cancer therapy would be appropriate in a specific patient.

Blinatumomab is a clinically approved therapy for acute lymphoblastic leukemia. As a bispecific CD19-directed CD3 T-cell engager, it is designed to bind to CD19 expressed on the surface of cancerous B cells and bind to CD3 expressed on the surface of T cells. This interaction stimulates immunological processes that results in the lysis of cancerous CD19+ B cells. Cytokine Release Syndrome (CRS), which may be life-threatening or fatal, has occurred in patients receiving blinatumomab. See BLINCYTO® (blinatumomab) product label, 2014.

Liu et al. report DNA-based nanoparticle tension sensors reveal that T-cell receptors transmit defined pN forces to their antigens for enhanced fidelity. PNAS, 2016, 113, 5610-5615.

Ma et al. report DNA probes that store mechanical information revealing transient piconewton forces applied by T cells. PNAS, 2019, 116 (34): 16949-16954. See also U.S. Patent App. No. 2021/0024985.

Ma et al. repot the magnitude of LFA-1/ICAM-1 forces fine-tune TCR-triggered T cell activation. Sci. Adv. 2022, 8, eabg4485, 16 pages.

Baek et al. report molecular tension probes to quantify cell-generated mechanical forces. Mol. Cells 2022, 45 (1): 26-32.

References cited herein are not an admission of prior art.

### SUMMARY

This disclosure contemplates methods of detecting, measuring, and/or quantifying a tensional force on cells that express an antigen. In certain embodiments, this disclosure relates to methods and devices disclosed herein used to determine or monitor whether a cancer or other immunotherapy is appropriate for a specific patient. In certain embodiments, the immunotherapy is administering blinatumomab or other bispecific engager to a patient.

In certain embodiments, the bispecific engager is a therapeutic agent such as an anticancer agent. In certain embodiments, the bispecific engager specifically binds a T cell specific antigen such as CD3, CD4, or CD8. In certain embodiments, the detected, measured, or quantified tensional force is used to diagnose whether a patient will be responsive to a therapy as detecting, measuring, or quantifying the tensional force is associated with the likelihood of the bispecific engager to be effective or not effective as a therapy, such as a chemotherapy.

In certain embodiments, methods and devices disclosed here are used to determine whether blinatumomab in not effective in a human patient due to insufficient T-cell forces/responses. In certain embodiments, methods and devices disclosed here are used to determine whether T cells of a patient are responses to blinatumomab prior to treatment.

In certain embodiments, methods and devices disclosed here are used to determine whether the likelihood of negative side effects is associated to higher-than-normal forces indicating high cytokine expression, e.g., a patient is at risk of cytokine release syndrome.

In certain embodiments, methods and devices disclosed here are used to determine whether and/or when an immunotherapy, e.g., blinatumomab therapy, cease to be effective or has limited efficacy. In certain embodiments, patients are diagnosed with sufficiently high levels of CD19+ cells/blasts and method disclosed herein indicate the patient has lower than normal T-cell responses using an immune therapy.

In certain embodiments, methods and devices disclosed here are used to monitor T cell responses, including alpha/beta T-cells, gamma/delta T-cells, NK cells, and other lymphocyte populations, in a patient on a repeated basis, e.g., weekly, biweekly, or monthly basis, to determine if the immunotherapy is productive or nonproductive.

In certain embodiments, methods and devices disclosed here are used to evaluate a test compound for its ability to simulate desirable therapeutic outcomes, e.g., by contacting a test compound with devices using methods disclosed herein and comparing the detection, measurement, or quantification to a normal or reference value indicating a desirable to undesirable attribute such as therapeutic efficacy. In certain embodiments, T-cell activity/stimulation is compared to antigen density, the presence of tumor infiltrating lymphocytes, and/or tumor growth rate.

In certain embodiments, methods and devices disclosed here are modified to present any antigen of interest and test real-time T cell activity in response to bispecific T-cell engagers by detecting, measuring, or quantifying a force readout as a biomarker that accounts for the physical cell-cell interactions in the cellular or tumor microenvironment.

In certain embodiments, methods and devices disclosed here are CAR-T cell immunotherapies wherein detection, measurements, and/or quantification of tensional force is associated with the intensity of a light and/or fluorescent signal which are used to assess the efficacy of T cells after they have been engineered to express a chimeric antigen receptor.

In certain embodiments, methods and devices disclosed here are used to predict safety, as CAR-T cells that exert extremely high forces put a patient at high risk for cytokine release syndrome. Thus, in certain embodiments, this disclosure contemplates methods of comparing a detected, measured, or quantified force, and making comparisons to a normal or reference value for determining whether a subject is at a high, normal, or low risk of cytokine release syndrome.

In certain embodiments, this disclosure relates to methods of detecting a light and/or fluorescent signal from a sample of a subject comprising a cell expressing an antigen comprising the steps of: a) exposing a device to a cell comprising a first antigen and a bi-specific engager that specifically binds the antigen (first) and specifically binds a second antigen; wherein the device comprises: i) a second antigen; ii) a nucleic acid complex linker having a first end and a second end, wherein the nucleic acid complex linker is linked to the second antigen at the first end; iii) a surface connected to the nucleic acid complex linker at the second end; iv) a quencher conjugated to the nucleic acid complex linker; and v) a fluorescent molecule conjugated to the nucleic acid complex linker, wherein the fluorescent molecule is configured to move its position relative to the quencher when the second antigen moves upon binding to the bi-specific engager bound to the first antigen on the cell; wherein the nucleic acid complex linker comprises a hairpin motif comprising a double stranded stem segment, a single stranded loop segment, a first end tail segment, and a second end tail segment; wherein the quencher and the first fluorescent molecule are configured to quench when the nucleic acid complex linker is in the form of a hairpin motif; wherein the quencher and the fluorescent molecule are not configured to quench when the nucleic acid complex linker is in the form of a single stranded motif, and wherein the bi-specific engager binds first antigen and the second antigen and pulls the second antigen away from the surface to unravel the hairpin motif into the single stranded motif removing the first fluorescent molecule from proximity to the quencher producing a light and/or fluorescent signal; and b) detecting the light and/or fluorescent signal.

In certain embodiments, the first antigen is CD3, CD4, or CD8.

In certain embodiments, the bi-specific engager has a domain that specifically binds CD3, CD4, or CD8.

In certain embodiments, the subject is receiving a chemotherapy treatment including a bi-specific T-cell engager that specifically binds both CD3 and the second antigen. In certain embodiments, the bi-specific T-cell engager is blinatumomab.

In certain embodiments, the detected light and/or fluorescent signal or lack of a detected light and/or fluorescent signal indicates an increased risk that the subject may not be responsive to the chemotherapy treatment or that the chemotherapy treatment has stopped working. In certain embodiments, a low light and/or fluorescent signal or lack of a light and/or fluorescent signal indicates that blinatumomab is not effective as a therapy in the subject, e.g., cancer treatment therapy, because blinatumomab is not able to sufficiently stimulate T cell responses.

In certain embodiments, the detected light and/or fluorescent signal indicates that a subject is experiencing cytokine release syndrome, or the subject is at an increased risk of cytokine release syndrome. In certain embodiments, a high or intense light and/or fluorescent signal indicates that blinatumomab is causing an excessive immune reaction.

In certain embodiments, the sample is from subject that has not yet received a chemotherapy treatment including a bi-specific T-cell engager, that is to be considered for such treatment, and/or thereafter is administered the bi-specific T-cell engager for treatment.

In certain embodiments, the second antigen is cluster of differentiation 19 (CD19), cluster of differentiation 10 (CD10), cluster of differentiation 20 (CD20), cluster of differentiation 33 (CD33), cluster of differentiation 38 (CD38), CD70 (tumor necrosis factor ligand superfamily member 7), CD133 (prominin 1), CD171 (L1 cell adhesion molecule), (EGFR) epidermal growth factor receptor, (HER2) human epidermal growth factor receptor 2, EGFR vIII (epidermal growth factor receptor variant 3) (MUC1) mucin1, (MUC16) mucin16, (EpCAM) epithelial cell adhesion molecule, (AFP) alpha-fetoprotein, (FAP) familial adenomatous polyposis, (CEA) carcinoembryonic antigen, (PSCA) prostate stem cell antigen, (PSMA) prostate-specific membrane antigen, (PSA) prostate-specific antigen, (AXL) AXL receptor tyrosine kinase, (DLL3) delta-like 3, (EPHA2) EPH receptor A2, (FRα) folate receptor alpha, (LMP1) Epstein-Barr virus latent membrane protein 1, (MAGE) melanoma antigen gene protein, MAGE-A1, MAGE-A3, MAGE-A4, (DR5) death receptor 5, (NKG2D) natural killer group 2 member D receptor, (CAIX) carbonic anhydrase IX, (TAG-72) tumor-associated glycoprotein 72, (GUCY2C) guanylate cyclase 2C, (ANTXR1) anthrax toxin receptor 1, (GSPG4) general secretion pathway protein G, (ROR) RAR-related orphan receptors, ROR1 (receptor tyrosine kinase like orphan receptor 1), IL13RA2 (Interleukin 13 Receptor Subunit Alpha 2), Wilms' tumor 1 (WT1), Survivin, Tn (aGalNAc-O-Ser/Thr), sialyl-Tn (aNeuAc2,6-aGalNAc-O-Ser/Thr), TF (bGal1,3-aGalNAc-O-Ser/Thr), CA 19-9 (Neu5Acα2-3Galβ1-3 [Fucα1-4]GlcNAcβ), Telomerase reverse transcriptase (TERT), Beta-hCG (Human chorionic gonadotropin), p53, Ras, bladder tumor antigen (BTA), antibody specific antigen Om5, GD2 (Ganglioside GD2), integrin alpha-v/beta-6, or mesothelin antigen. BCMA (TNF receptor superfamily member 17\B-cell maturation protein), CD123 (interleukin 3 receptor subunit α\CD123 antigen), CD138 (syndecan 1), CD22 (SIGLEC2), CD5 (lymphocyte antigen T1/Leu-1), Ig kappa chain, LeY (fucosyltransferase 3/Lewis Blood Group), NKG2D ligand (killer cell lectin like receptor K1/CD314), WT1 (Wilms' tumor antigen 1), C-Met (MET proto-oncogene), CAIX (carbonic anhydrase 9), GPC3 (glypican 3), HPV16-E6 (human papillomavirus E6 protein), MARTI (melan-A), NY-ESO-1 (cancer/testis antigen 1B), PD-L1 (CD274 molecule), PSMA (folate hydrolase 1), or VEGFR2 (kinase insert domain receptor/vascular endothelial growth factor receptor 2).

In certain embodiments, this disclosure relates to methods of detecting a signal from a sample of a subject comprising a cell expressing an antigen comprising the steps of: a) exposing a device to a cell comprising a first antigen and a bi-specific engager that specifically binds the first antigen and a second antigen in the presence of a locking oligonucleotide; wherein the device comprises: i) a second antigen; ii) a nucleic acid complex linker having a first end and a second end, wherein the nucleic acid complex linker is linked to the second antigen at the first end; iii) a surface connected to the nucleic acid complex linker at the second end; iv) a quencher conjugated to the nucleic acid complex linker; and v) a fluorescent molecule conjugated to the nucleic acid complex linker, wherein the fluorescent molecule is configured to move its position relative to the quencher when the second antigen moves upon binding to the bi-specific engager bound to the first antigen on the device; wherein the nucleic acid complex linker comprises a hairpin motif comprising a double stranded stem segment, a single stranded loop segment, a first end tail segment, and a second end tail segment; wherein the quencher and the first fluorescent molecule are configured to quench when the nucleic acid complex linker is in the form of a hairpin motif; wherein the quencher and the fluorescent molecule are not configured to quench when the nucleic acid complex linker is in the form of a single stranded motif; and wherein the bi-specific engager binds first antigen and the second antigen and pulls the second antigen away from the surface to unravel the hairpin motif into the single stranded motif removing the first fluorescent molecule from proximity to the quencher producing a light and/or fluorescent signal and the locking oligonucleotide hybridizes to the single stranded motif under conditions such that the nucleic acid complex linker is locked in an extended form derived from the single stranded motif; and b) detecting the light and/or fluorescent signal.

In certain embodiments, the locking oligonucleotide comprises a sequence that is only one nucleotide that base pairs with the last nucleotide of the double stranded stem segment followed by the reverse complement of the single stranded loop segment followed by the reverse complement of the double stranded stem segment.

In certain embodiments, this disclosure relates to methods of detecting a light and/or fluorescent signal from a sample of a subject comprising a cell expressing a CD3 antigen comprising the steps of: a) exposing a device to a cell comprising CD3 antigen and a bi-specific T-cell engager that specifically binds a CD3 antigen and a second antigen; wherein the device comprises: i) a second antigen; ii) a nucleic acid complex linker having a first end and a second end, wherein the nucleic acid complex linker is linked to the second antigen at the first end; iii) a surface connected to the nucleic acid complex linker at the second end; iv) a quencher conjugated to the nucleic acid complex linker; and v) a first fluorescent molecule conjugated to the nucleic acid complex linker wherein the fluorescent molecule is configured to move its position relative to the quencher when the second antigen moves upon binding to the bi-specific T-cell engager, bound to CD3 on the device; wherein the nucleic acid complex linker comprises a hairpin motif comprising a double stranded stem segment, a single stranded loop segment, a first end tail segment, and a second end tail segment; wherein the quencher and the first fluorescent molecule are configured to quench when the nucleic acid complex linker is in the form of a hairpin motif; wherein the quencher and the first fluorescent molecule are not configured to quench when the nucleic acid complex linker is in the form of a single stranded motif; and wherein the bi-specific T-cell engager binds CD3 and the second antigen and pulls the second antigen away from the surface to unravel the hairpin motif into the single stranded motif removing the first fluorescent molecule from proximity to the quencher producing a light and/or fluorescent signal; and b) detecting the light and/or fluorescent signal.

In certain embodiments, this disclosure relates to methods of detecting a light and/or fluorescent signal from a blood sample of a subject comprising a cell expressing a CD3 antigen comprising the steps of: a) exposing a device to a cell comprising CD3 antigen and a bi-specific T-cell engager that specifically binds a CD3 antigen and a second antigen in the presence of a locking oligonucleotide; wherein the device comprises: i) a second antigen; ii) a nucleic acid complex linker having a first end and a second end, wherein the nucleic acid complex linker is linked to the second antigen at the first end; iii) a surface connected to the nucleic acid complex linker at the second end; iv) a quencher conjugated to the nucleic acid complex linker; and v) a first fluorescent molecule conjugated to the nucleic acid complex linker wherein the fluorescent molecule is configured to move its position relative to the quencher when the second antigen moves upon binding to the bi-specific T-cell engager, bound to CD3 on the device; wherein the nucleic acid complex linker comprises a hairpin motif comprising a double stranded stem segment, a single stranded loop segment, a first end tail segment, and a second end tail segment; wherein the quencher and the first fluorescent molecule are configured to quench when the nucleic acid complex linker is in the form of a hairpin motif; wherein the quencher and the first fluorescent molecule are not configured to quench when the nucleic acid complex linker is in the form of a single stranded motif; and wherein the bi-specific T-cell engager binds CD3 and the second antigen and pulls the second antigen away from the surface to unravel the hairpin motif into the single stranded motif removing the first fluorescent molecule from proximity to the quencher producing a light and/or fluorescent signal and the locking oligonucleotide hybridizes to the single stranded motif under conditions such that the nucleic acid complex linker is locked in an extended form derived from the single stranded motif; and b) detecting the light and/or fluorescent signal.

In certain embodiments, this disclosure relates to methods of preventing cytokine release syndrome comprising obtaining a sample of T cells or other immune cells from a patient diagnosed with cancer; contacting the T cells with blinatumomab and a device comprising: a nucleic acid complex linker having a first end and a second end, wherein the nucleic acid complex linker is linked to CD19 antigen at the first end; a surface connected to the nucleic acid complex linker at the second end; a quencher conjugated to the nucleic acid complex linker; and a fluorescent molecule conjugated to the nucleic acid complex linker, wherein the nucleic acid complex linker comprises a hairpin motif comprising a double stranded stem segment, a single stranded loop segment, a first end tail segment, and a second end tail segment; wherein the quencher and the first fluorescent molecule are configured to quench when the nucleic acid complex linker is in the form of a hairpin motif; wherein the quencher and the first fluorescent molecule are not configured to quench when the nucleic acid complex linker is in the form of a single stranded motif; wherein the fluorescent molecule is in a position and configured to move the position of the fluorescent molecule away from the quencher relative to the quencher when the CD19 antigen moves upon binding to a blinatumomab providing a detected light and/or fluorescent signal; comparing the detected light and/or fluorescent signal to normal or reference light and/or fluorescent signal indicative of a high risk of cytokine release syndrome, wherein if the detected light and/or fluorescent signal is the same or similar or higher than a normal or reference light and/or fluorescent signal it is indicative of a high risk of cytokine release syndrome.

In certain embodiments, the detected light and/or fluorescent signal is recorded on a computer readable medium providing a quantitative result.

In certain embodiments, methods further comprise comparing the detected light and/or fluorescent signal to normal or reference light and/or fluorescent signal.

In certain embodiments, methods further comprise comparing the detected light and/or fluorescent signal to normal or reference light and/or fluorescent signal which is done on a computer providing a comparative result.

In certain embodiments, methods further comprise communicating to the patient or a medical professional the quantitative result, the comparative result, or that the patent is at a high risk of cytokine release syndrome.

In certain embodiments, methods further comprise diagnosing the patient with a high risk of cytokine release syndrome if administered blinatumomab.

In certain embodiments, this disclosure relates to devices comprises: i) an antigen found on cancer cells, immune cells, non-immune cells; ii) a nucleic acid complex linker having a first end and a second end, wherein the nucleic acid complex linker is linked to the antigen at the first end; iii) a surface connected to the nucleic acid complex linker at the second end; iv) a quencher conjugated to the nucleic acid complex linker; and v) a first fluorescent molecule conjugated to the nucleic acid complex linker wherein the fluorescent molecule is configured to move its position relative to the quencher when the antigen moves upon binding to a receptor of the antigen; wherein the nucleic acid complex linker comprises a hairpin motif comprising a double stranded stem segment, a single stranded loop segment, a first end tail segment, and a second end tail segment; wherein the quencher and the first fluorescent molecule are configured to quench when the nucleic acid complex linker is in the form of a hairpin motif; and wherein the quencher and the first fluorescent molecule are not configured to quench when the nucleic acid complex linker is in the form of a single stranded motif.

In certain embodiments, this disclosure contemplates devices comprising: i) a CD19 antigen; ii) a nucleic acid complex linker having a first end and a second end, wherein the nucleic acid complex linker is linked to the CD19 antigen at the first end; iii) a surface connected to the nucleic acid complex linker at the second end; iv) a quencher conjugated to the nucleic acid complex linker; and v) a first fluorescent molecule conjugated to the nucleic acid complex linker wherein the fluorescent molecule is configured to move its position relative to the quencher when the CD19 antigen moves upon binding to a receptor of a bi-specific T-cell engager bound to CD3, wherein the nucleic acid complex linker comprises a hairpin motif comprising a double stranded stem segment, a single stranded loop segment, a first end tail segment, and a second end tail segment; wherein the quencher and the first fluorescent molecule are configured to quench when the nucleic acid complex linker is in the form of a hairpin motif; and wherein the quencher and the first fluorescent molecule are not configured to quench when the nucleic acid complex linker is in the form of a single stranded motif.

### BRIEF DESCRIPTION OF THE SEVERAL VIEWS OF THE DRAWINGS

FIGS. 1A-1C show data indicating effector functions are compromised in murine CD4+ and CD8+ T-cells isolated from obese mice.

FIG. 1A shows an illustration of isolating T-cells from lean and obese mice and activating them with an anti-CD3 and anti-CD28 antibodies for further separation and analysis of cellular properties.

FIG. 1B shows data on the percent of CD4+ and CD8+OT-1 T-cells expressing interferon gamma (IFN-γ), tumor necrosis factor-alpha (TNF-alpha), perforin, and granzyme B after 72 h of activation.

FIG. 1C shows data indicating T-cell function is compromised in mice with obesity. T-cell isolated from mice with obesity exhibit reductions spreading on glass slides and in the actin/T-cell signaling adaptor protein WAVE1.

FIG. 2A shows a schematic of an α-CD3ε-presenting DNA-based tension probe used to detect a 4.7 pN tension signal exerted by polyclonal CD4+ and CD8+ T-cells treated with UCM, ACM, or SCM.

FIG. 2B shows schematic of a DNA-based tension probe presenting the peptide SIINFEKL (SEQ ID NO: 1) OVA-pMHC bound to antigen-specific OT-1 T cells wherein images were used to quantify a 4.7 pN tension signal exerted by OT-1 T cells cultured with UCM, ACM or SCM.

FIG. 2C shows DNA oligonucleotide sequences used for creating the DNA tension probes including the names, sequences (SEQ ID NOs: 2-7), and chemical modifications of DNA oligonucleotides.

FIG. 2D illustrated one method of making tension probes disclosed herein on glass coverslips using gold nanoparticles with functionalized DNA-based tension probe surfaces.

FIG. 2E shows quantification data based on images using 4.7 pN tension probes exposed to polyclonal CD4+ and CD8+ T-cells treated with UCM, ACM, or SCM for 24.

FIG. 2F shows quantification data based on images using 4.7 pN tension probes exposed to OT-1 T cells treated with UCM, ACM, or SCM for 24 h.

FIG. 2G shows fluorescent intensity data on T-cells isolated from mice with obesity which exhibited reduction in spreading and TCR-mediated biomechanical forces tested using 4.7 pN tension probes. Naive T-cells were isolated from lean and obese mice. Force measurements were determined using 4.7 pN anti-CD3 tension probes and the spreading area was determined using microscopy.

FIG. 3A illustrates the structure of blinatumomab and binding domains. Blinatumomab is a bispecific T-cell engager (BiTE) that bridges CD3+ T-cells with CD19+ B-cells to induce cytolysis.

FIG. 3B illustrates blinatumomab inducing cell lysis by connecting cytotoxic T-cells to B-ALL cells.

FIG. 3C illustrates blinatumomab interreacting to bridge CD3+ T-cells with CD19 presenting a signal illuminating probe with a locking strand to maintain a signal.

FIGS. 4A-4B shows data on blinatumomab-mediated killing of malignant B-cells. Cytometer images were evaluated to monitor blinatumomab-mediated immunological synapse formation after a 24 h treatment with UCM, SCM or ACM. T-cells were stained with LAMP-1, LFA-2, and SirActin™ as markers of immunological synapse formation. Nalm6 B-cells were stained with cell tracker violet.

FIG. 4A shows data on the fold increase of immunological synapse formation of blinatumomab-treated samples compared to vehicle-treated samples.

FIG. 4B shows data on blinatumomab-mediated cytolysis of Nalm6 B-cells after a 24 h treatment with UCM (right), left shows data after 48 h treatment.

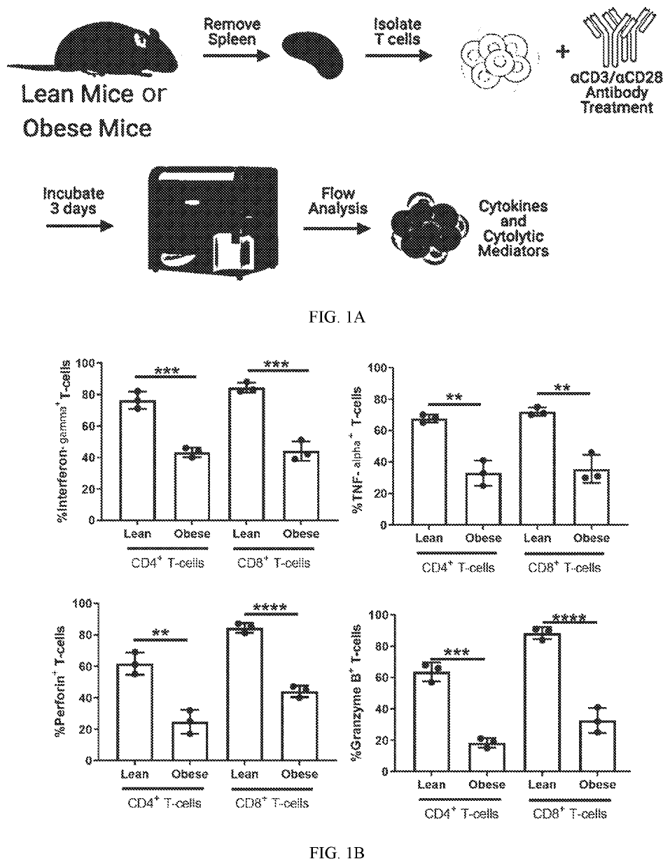

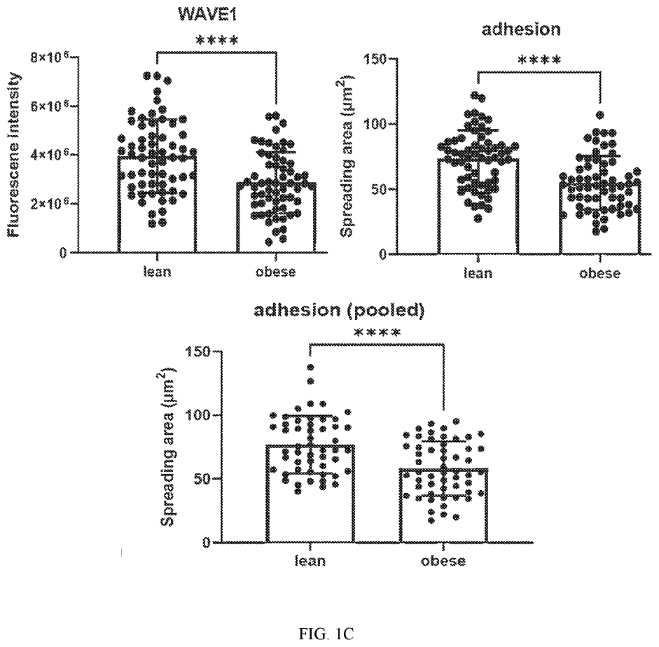

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

An “embodiment” of this disclosure refers to an example, but not necessarily limited to such example. As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature. It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps. The term “comprising” in reference to an oligonucleotide having a nucleic acid sequence refers to an oligonucleotide that may contain additional 5′ (5′ terminal end) or 3′ (3′ terminal end) nucleotides, i.e., the term is intended to include the oligonucleotide sequence within a larger nucleic acid. “Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods, compared to those of the corresponding compositions or methods disclosed herein. The term “consisting of” in reference to an oligonucleotide having a nucleotide sequence refers an oligonucleotide having the exact number of nucleotides in the sequence and not more or having not more than a range of nucleotide expressly specified in the claim. For example, “5′ sequence consisting of” is limited only to the 5′ end, i.e., the 3′ end may contain additional nucleotides. Similarly, a “3′ sequence consisting of” is limited only to the 3′ end, and the 5′ end may contain additional nucleotides.

As used herein, the term “conjugated” refers to linking molecular entities through covalent bonds, or by other specific binding interactions, such as due to hydrogen bonding or other van der Walls forces. The force to break a covalent bond is high, e.g., about 1500 pN for a carbon-to-carbon bond. The force to break a combination of strong protein interactions is typically a magnitude less, e.g., biotin to streptavidin is about 150 pN. Thus, a skilled artisan would understand that conjugation must be strong enough to restrict the breaking of bonds in order to implement the intended results. In certain embodiments, the term conjugated is intended to include linking molecular entities that do not break unless exposed to a force of about greater than about 5, 10, 25, 50, 75, 100, 125, or 150 pN depending on the context.

A “linking group” refers to any variety of molecular arrangements that can be used to bridge or conjugate molecular moieties together. An example formula may be —Rₙ— wherein R is selected individually and independently at each occurrence as: —CRₙRₙ—, —CHRₙ—, —CH—, —C—, —CH₂—, —C(OH)Rₙ, —C(OH)(OH)—, —C(OH)H, —C(Hal)Rₙ—, —C(Hal)(Hal)-, —C(Hal)H—, —C(N₃)Rₙ—, —C(CN)Rₙ—, —C(CN)(CN)—, —C(CN) H—, —C(N₃)(N₃)—, —C(N₃) H—, —O—, —S—, —N—, —NH—, —NRₙ—, —(C═O)—, —(C═NH)—, —(C═S)—, —(C═CH₂)—, which may contain single, double, or triple bonds individually and independently between the R groups. If an R is branched with an Rₙ it may be terminated with a group such as —CH₃, —H, —CH═CH₂, —CCH, —OH, —SH, —NH₂, —N₃, —CN, or -Hal, or two branched Rs may form an aromatic or non-aromatic cyclic structure. It is contemplated that in certain instances, the total Rs or “n” may be less than 100 or 50 or 25 or 10. Examples of linking groups include bridging alkyl groups, alkoxyalkyl, polyethylene glycols, amides, esters, and aromatic groups.

As used herein, the terms “oligonucleotide” is meant to include nucleic acids, ribonucleic or deoxyribonucleic acids, mixtures, nucleobase polymers, or analogs thereof. An oligonucleotide can include native or non-native bases. In this regard, a native deoxyribonucleic acid can have one or more bases selected from the group consisting of adenine, thymine, cytosine or guanine and a ribonucleic acid can have one or more bases selected from the group consisting of uracil, adenine, cytosine, or guanine. It will be understood that a deoxyribonucleic acid used in the methods or compositions set forth herein can include uracil bases and a ribonucleic acid can include a thymine base.

The term “nucleobase polymer” refers to nucleic acids and chemically modified forms with nucleobase monomers. In certain embodiments, methods and compositions disclosed herein may be implemented with nucleobase polymers comprising units of a ribose, 2′deoxyribose, locked nucleic acids (1-(hydroxymethyl)-2,5-dioxabicyclo[2.2.1]heptan-7-ol), 2′-O-methyl groups, a 3′-3′-inverted thymidine, phosphorothioate linkages, or combinations thereof. In certain embodiments, the nucleobase polymer may be less than 100, 50, or 35 nucleotides or nucleobases.

Nucleobase monomers are nitrogen containing aromatic or heterocyclic bases that bind to naturally occurring nucleic acids through hydrogen bonding otherwise known as base pairing. A typical nucleobase polymer is a nucleic acid, RNA, DNA, or chemically modified form thereof. A nucleobase polymer may be single or double stranded or both, e.g., they may contain overhangs. Nucleobase polymers may contain naturally occurring or synthetically modified bases and backbones. In certain embodiments, a nucleobase polymer need not be entirely complementary, e.g., may contain one or more insertions, deletions, or be in a hairpin structure provided that there is sufficient selective binding.

With regard to the nucleobases, it is contemplated that the term encompasses isobases, otherwise known as modified bases, e.g., are isoelectronic or have other substitutes configured to mimic naturally occurring hydrogen bonding base-pairs, e.g., within any of the sequences herein U may be substituted for T, or T may be substituted for U. Examples of nucleotides with modified adenosine or guanosine include, but are not limited to, hypoxanthine, xanthine, 7-methylguanine. Examples of nucleotides with modified cytidine, thymidine, or uridine include 5,6-dihydrouracil, 5-methylcytosine, 5-hydroxymethylcytosine. Contemplated isobases include 2′-deoxy-5-methylisocytidine (iC) and 2′-deoxy-isoguanosine (iG) (see U.S. Pat. Nos. 6,001,983; 6,037,120; 6,617,106; and 6,977,161).

Nucleobase polymers may be chemically modified, e.g., within the sugar backbone or on the 5′ or 3′ ends. As such, in certain embodiments, nucleobase polymers disclosed herein may contain monomers of phosphodiester, phosphorothioate, methylphosphonate, phosphorodiamidate, piperazine phosphorodiamidate, ribose, 2′-O-methy ribose, 2′-O-methoxyethyl ribose, 2′-fluororibose, deoxyribose, 1-(hydroxymethyl)-2,5-dioxabicyclo[2.2.1]heptan-7-ol, P-(2-(hydroxymethyl) morpholino)-N,N-dimethylphosphon amidate, morpholin-2-ylmethanol, (2-(hydroxymethyl) morpholino)(piperazin-1-yl)phosphinate, or peptide nucleic acids or combinations thereof.

In certain embodiments, the nucleobase polymers contain biotin or alkyne modifications or modified nucleobases or conjugated to other moieties using linking groups and conjugating chemistry such as the reaction of azido and alkyne groups to form triazole linking groups.

In certain embodiments, the nucleotide base polymer is single or double stranded and/or is 3′ end capped with one, two, or more thymidine nucleotides and/or 5′ end polyphosphorylated, e.g., di-phosphate, tri-phosphate. In certain embodiments, nucleobase polymers have 3′ and 5′ end poly T spacers.

In certain embodiments, the nucleobase polymer can be modified to contain a phosphodiester bond, methylphosphonate bond, or phosphorothioate bond. The nucleobase polymers can be modified with, for example, 2′-amino, 2′-O-allyl, 2′-fluoro, 2′-O-methyl, on the ribose ring. Constructs can be purified by gel electrophoresis using general methods or can be purified by high pressure liquid chromatography and re-suspended in water.

In certain embodiments, nucleobase polymers include one or more (e.g., about 1, 2, 3, 4, 5, 6, 7, 8, 9, 10, or more) LNA “locked nucleic acid” nucleotides such as a 2′,4′-C methylene bicyclo nucleotide (see for example U.S. Pat. Nos. 6,639,059, 6,670,461, U.S. Pat. No. 7,053,207).

In one embodiment, the disclosure features modified nucleobase polymers, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl substitutions.

As used herein, the term “surface” refers to the outside part of an object. The area is typically of greater than about one hundred square nanometers, one square micrometer, or more than one square millimeter. Examples of contemplated surfaces are on a particle, bead, wafer, array, well, microscope slide, transparent or opaque glass, polymer, or metal, or the bottom of a zero-mode waveguide. A “zero-mode waveguide (ZMW)” refers to a confined structure or chamber located in an opening, e.g., hole, of a metal film deposited on a transparent substrate. See Levene et al., Science, 2003, 299:682-686. The chamber acts as a wave guide for light coming out of the bottom of the opening. The openings are typically about 150-50 nm in width and depth. Due to the behavior of light when it travels through a small aperture, the optical field decays exponentially inside the chamber. Thus, fluorescent molecules will lose fluorescence as they move away from the bottom of the chamber. In certain embodiments, imaging chambers have a width, diameter, depth of more than 150 nanometers to 1 micrometer, or between 1 micrometer to 1 millimeter, or between 1 millimeter to 1 centimeter, or between 1 centimeter to 10 centimeter, or larger.

As used herein, a “subject” refers to any animal, preferably a human patient, livestock, or domestic pet.

In certain embodiments, methods disclosed herein include measurements that are compared to a normal or reference value. As used herein, a “reference value” can be an absolute value; a relative value; an average value; a median value, a mean value, or a value as compared to a particular control or baseline value. A reference value can be based on an individual sample or a large number of samples, such as from patients or normal individuals.

A “normalized measured” value refers to a measurement taken and adjusted to take background into consideration. Background subtraction to obtain total fluorescence is considered a normalized measurement. The background subtraction allows for the correction of background fluorescence that is inherent in the optical system and assay buffers.

Unless stated otherwise as apparent from the following discussion, it will be appreciated that terms such as “detecting,” “receiving,” “quantifying,” “mapping,” “generating,” “registering,” “determining,” “obtaining,” “processing,” “computing,” “deriving,” “estimating,” “calculating,” “inferring” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods may be compiled for execution on a variety of hardware platforms and for interface to a variety of operating systems. In addition, embodiments are not described with reference to any particular programming language. It will be appreciated that a variety of programming languages may be used to implement embodiments of the disclosure.

In some embodiments, the disclosed methods may be implemented using software applications that are stored in a memory and executed by a processor (e.g., CPU) provided on the system. In some embodiments, the disclosed methods may be implanted using software applications that are stored in memories and executed by CPUs distributed across the system. As such, the modules of the system may be a general purpose computer system that becomes a specific purpose computer system when executing the routine of the disclosure. The modules of the system may also include an operating system and micro instruction code. The various processes and functions described herein may either be part of the micro instruction code or part of the application program or routine (or combination thereof) that is executed via the operating system.

It is to be understood that the embodiments of the disclosure may be implemented in various forms of hardware, software, firmware, special purpose processes, or a combination thereof. In one embodiment, the disclosure may be implemented in software as an application program tangible embodied on a computer readable program storage device. The application program may be uploaded to, and executed by, a machine comprising any suitable architecture. The system and/or method of the disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

It is to be further understood that because some of the constituent system components and method steps depicted in the accompanying figures may be implemented in software, the actual connections between the systems components (or the process steps) may differ depending upon the manner in which the disclosure is programmed. Given the teachings of the disclosure provided herein, one of ordinary skill in the related art will be able to contemplate these and similar implementations or configurations of the disclosure.

As used herein, the term “bispecific engager” refers to an organic molecule, i.e., substantially comprised of carbon, hydrogen, and oxygen, e.g., peptide(s), protein(s), antibodies, fragments, that specifically binds to two antigens, e.g., wherein the two antigens are not the same organic molecules. Typically, one of the antigens is an antigen which is more highly or exclusively expressed on a cancer cell, e.g., a surface antigen associated with cancerous cell, and the other antigen is an immune cell associated antigen, a surface antigen associated with cells of the immune system or modified immune cells that express a receptor that binds antigens commonly found on cancer cells in order to engage cancer cells, e.g., to eliminate or kill.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term “specific binding agent” refers to a molecule, such as a proteinaceous molecule, that binds a target molecule with a greater affinity than other random molecules or proteins. Examples of specific binding agents include an antibody that bind an epitope of an antigen or a receptor which binds a ligand. In certain embodiments, “Specifically binds” refers to the ability of a specific binding agent (such as an ligand, receptor, enzyme, antibody or binding region/fragment thereof) to recognize and bind a target molecule or polypeptide, such that its affinity (as determined by, e.g., affinity ELISA or other assays) is at least 10 times as great, but optionally 50 times as great, 100, 250 or 500 times as great, or even at least 1000 times as great as the affinity of the same for any other or other random molecule or polypeptide.

As used herein, the term “ligand” refers to an organic molecule, i.e., substantially comprised of carbon, hydrogen, and oxygen, that binds a “receptor.” Receptors are organic molecules typically found on the surface of a cell. Through binding a ligand to a receptor, the cell has a signal of the extra cellular environment which may cause changes inside the cell. As a convention, a ligand is usually used to refer to the smaller of the binding partners from a size standpoint, and a receptor is usually used to refer to a molecule that spatially surrounds the ligand or portion thereof. However as used herein, the terms can be used interchangeably as they generally refer to molecules that are specific binding partners. For example, a glycan may be expressed on a cell surface glycoprotein and a lectin may bind the glycan. As the glycan is typically smaller and surrounded by the lectin during binding, it may be considered a ligand even though it is a receptor of the lectin binding signal on the cell surface. In another example, a double stranded oligonucleotide sequence contains two complimentary nucleic acid sequences. Either of the single stranded sequences may be consider the ligand or receptor of the other. In certain embodiments, a ligand is contemplated to be a compound that has a molecular weight of less than 500 or 1,000. In certain embodiments, a receptor is contemplated to be a compound that has a molecular weight of greater than 2,000 or 5,000. In any of the embodiments disclosed herein the position of a ligand and a receptor may be switched.

In certain contexts, an “antibody” refers to a protein-based molecule that is naturally produced by animals in response to the presence of a protein or other molecule or that is not recognized by the animal's immune system to be a “self” molecule, i.e., recognized by the animal to be a foreign molecule, i.e., an antigen to the antibody. The immune system of the animal will create an antibody to specifically bind the antigen, and thereby targeting the antigen for degradation or elimination, or any cell or organism attached to the antigen. It is well recognized by skilled artisans that the molecular structure of a natural antibody can be synthesized and altered by laboratory techniques. Recombinant engineering can be used to generate fully synthetic antibodies or fragments thereof providing control over variations of the amino acid sequences of the antibody. Thus, the term “antibody” is intended to include natural antibodies, monoclonal antibody, or non-naturally produced synthetic antibodies, such as specific binding single chain antibodies, bispecific antibodies, or fragments thereof. These antibodies may have chemical modifications. The term “monoclonal antibodies” refers to a collection of antibodies encoded by the same nucleic acid molecule that are optionally produced by a single hybridoma (or clone thereof) or other cell line, or by a transgenic mammal such that each monoclonal antibody will typically recognize the same antigen. The term “monoclonal” is not limited to any particular method for making the antibody, nor is the term limited to antibodies produced in a particular species, e.g., mouse, rat, etc.

In humans, from a structural standpoint, an antibody is a combination of proteins: two heavy chain proteins and two light chain proteins. Alternatively, other animal produce antibodies from nucleic acids that encode a single protein. In humans, the heavy chains are longer than the light chains. The two heavy chains typically have the same amino acid sequence. Similarly, the two light chains typically have the same amino acid sequence. Each of the heavy and light chains contain a variable segment that contains amino acid sequences which participate in binding to the antigen. The variable segments of the heavy chain do not have the same amino acid sequences as the light chains. The variable segments are often referred to as the antigen binding domains. The antigen and the variable regions of the antibody may physically interact with each other at specific smaller segments of an antigen often referred to as the “epitope.” Epitopes usually consist of surface groupings of molecules, for example, amino acids or carbohydrates. The terms “variable region,” “antigen binding domain,” and “antigen binding region” refer to that portion of the antibody molecule which contains the amino acid residues that interact with an antigen and confer on the antibody its specificity and affinity for the antigen. Small binding regions within the antigen-binding domain that typically interact with the epitope are also commonly alternatively referred to as the “complementarity-determining regions, or CDRs.”

A “chimeric antibody” is a molecule in which different portions of the antibody are derived from different immunoglobulin molecules such that the entire molecule is not naturally occurring. Examples of chimeric antibodies include those having a variable region derived from a non-human antibody and a human immunoglobulin constant region. The term is also intended to include antibodies having a variable region derived from one human antibody grafted to an immunoglobulin constant region of a predetermined sequences or the constant region from another human for which there are allotypic differences residing in the constant regions of any naturally occurring antibody having the variable regions, e.g., CDRs 1, 2, and 3 of the light and heavy chain. Human heavy chain genes exhibit structural polymorphism (allotypes) that are inherited as a haplotype. The serologically defined allotypes differ within and between population groups. See Jefferis et al. mAb, 1 (2009), pp. 332-338.

“Single chain antibodies” refer to a single peptide containing naturally or non-naturally occurring sequences, including synthetically modified peptide sequences, derived from an antibody variable region that specifically binds an antigen of interest. Single chain antibodies are sometimes fragments or variants of naturally occurring mammalian antibodies. Such antibodies are sometimes referred to as single-domain antibodies (sdAbs or VHHs), or camelid single-domain antibodies, e.g., when derived from an animal of Camelidae family, e.g., lamas, camels.

A “heterologous” nucleic acid sequence or peptide sequence refers to a nucleic acid sequence or a peptide sequence that does not naturally occur, e.g., because the whole sequence contains a segment from other plants, bacteria, viruses, other organisms, or joinder of two sequences that occur the same organism but are joined together in a manner that does not naturally occur in the same organism or any natural state.

As used herein, a “chimeric antigen receptor” or “CAR” refers to a protein receptor, which introduces an antigen specificity, via an antigen binding domain, onto cells (immune cells) to which it is expressed (for example T cells such as naive T cells, central memory T cells, effector memory T cells or combination thereof) thus combining the antigen binding properties of the antigen binding domain with the T cell activity (e.g. lytic capacity and self-renewal) of T cells. A CAR typically includes an extracellular antigen-binding domain (ectodomain), a transmembrane domain and an intracellular signaling domain. The intracellular signaling domain generally contains at least one immunoreceptor tyrosine-based activation motif (ITAM) signaling domain, e.g., derived from CD3zeta, and optionally at least one costimulatory signaling domain, e.g. derived from CD28 or 4-1BB.

In order to improve the ability of immune cells to kill cancerous cells, T cells can be isolated from the blood of a patient and genetically altered to express chimeric antigen receptors (CARs) to specifically target proteins expressed on the surface of cancerous cells and stimulate an immune response. When put back into the patient, the cells attack the cancerous cells. Brentjens et al. report that T cells altered to bind CD19 can induce remissions of cancer in adults with chemotherapy-refractory acute lymphoblastic leukemia. Sci Transl Med, 2013, 5 (177): 177ra38.

Whole blood is composed of plasma, red blood cells (RBCs; or erythrocytes), platelets, and nucleated white blood cells, also referred to as leukocytes. The leukocytes can be further categorized into mononuclear cells and polymorphonuclear cells (or granulocytes). There are different techniques to obtain peripheral blood mononuclear cells (PBMCs), polymorphonuclear cells, leukocytes, or specific cell subsets, e.g., isolate specific cells directly by using flow cytometry, depleting red blood cells, centrifugation, and/or apheresis.

In a typical procedure, T cells and other immune cells are purified and isolated from blood or bone marrow. For example, T cells are collected via apheresis, a process that withdraws blood from the body and removes one or more blood components (such as plasma, platelets, or other white blood cells). The remaining blood is then returned back into the body. The cells are exposed to a recombinant vector, such as a lentiviral vector, that infects the cells in a way that a recombinant chimeric antigen receptor (CAR) protein is produced and presented in the cell membrane.

Before and/or after infecting the isolated cells with the recombinant vector, the cells may be induced to replicate. The genetically modified T cells may be expanded by growing cells in the laboratory until there are sufficient number of them. Optionally, these CAR T cells are frozen. The modified cells are then administered back to the patient. Various T cell subsets, as well as T cell progenitors and other immune cells such as natural killer (NK) cells, macrophages, or monocytes can be targeted with a CAR.

In certain embodiments, the targeting sequence in a chimeric antigen receptor refers to any variety of polypeptide sequences capable of selectively binding to a targeted associated molecule. The targeting sequences may be derived from variable binding regions of antibodies, single chain antibodies, and antibody mimetics. In certain embodiments, targeting sequence is a single-chain variable fragment (scFv) derived from an antibody. The targeting sequence is typically connected to intracellular domains by a hinge/transmembrane region, commonly derived from CD8 or IgG4. The intracellular domains may contain co-stimulatory domains such as CD80, CD86, 4-1BBL, OX40L and CD70 and/or CD28 linked to the cytoplasmic signaling domain of CD3zeta. See Sadelain et al. The basic principles of chimeric antigen receptor (CAR) design, Cancer Discov. 2013, 3 (4): 388-398.

Peripheral blood mononuclear cells (PBMCs) may be isolated by leukapheresis. T cells can be enriched by mononuclear cells counter-flow elutriation and expanded by addition of anti-CD3/CD28 antibody coated paramagnetic beads for activation of T cells. Cells may be expanded, harvested, and cryopreserved in infusible medium sometime after the subject has had an autologous or allogeneic stem-cell transplantation.

Cells may be obtained by isolation from peripheral blood and optionally purified by fluorescent activated cells sorting e.g., mixing cells with fluorescent antibodies or other fluorescent agents (molecular beacons) and separating the cells by flow cytometry based fluorescent sorting. Another option for cells sorting is to provide magnetic particles that are conjugated to specific binding agents, such as antibodies against a particular antigen on a target cells surface. After mixing with a sample, the antibody bound cells are put through a purification column containing a matrix composed of ferromagnetic spheres. When placed on a magnetic separator, the spheres amplify the magnetic field. The unlabeled cells pass through while the magnetically labeled cells are retained within the column. The flow-through can be collected as the unlabeled cells fraction. After a short washing step, the column is removed from the separator, and the magnetically labeled cells are eluted from the column.

CD3 is expressed on T cells as it is associated with the T cells receptor (TCR). The majority of TCR are made up of alpha beta chains (alpha beta T-cells). Alpha beta T-cells typically become double-positive intermediates (CD4+CD8+) which mature into single-positive (CD4+CD8−) T helper cells or (CD4−CD8+) cytotoxic T cells. T helper cells interact with antigen presenting dendritic cells and B cells. Upon activation with cognate antigen by dendritic cells, antigen specific CD4 T cells can differentiate to become various types of effector CD4 T cells with specific roles in promoting immune responses.

T cells may be isolated and separated from a human sample (blood or PBMCs or bone marrow) based on the expression of alpha beta T cells receptor (TCR), gamma delta T cells receptor, CD2, CD3, CD4, CD8, CD4 and CD8, NK1.1, CD4 and CD25 and other combinations based on positive or negative selection. In certain embodiments, the immune cells are CD8+, CD4+, alpha beta T cells, delta gamma T cells, natural killer cells and/or double-negative alpha beta T cells, macrophages, NK T cells, and cells derived from umbilical cord blood, bone marrow, or peripheral blood from human samples.

In certain embodiments, this disclosure relates to methods of preventing cytokine release syndrome comprising obtaining a sample of T cells or other immune cells from a patient diagnosed with cancer; contacting the T cells with blinatumomab and a device comprising: a nucleic acid complex linker having a first end and a second end, wherein the nucleic acid complex linker is linked to CD19 antigen at the first end; a surface connected to the nucleic acid complex linker at the second end; a quencher conjugated to the nucleic acid complex linker; and a fluorescent molecule conjugated to the nucleic acid complex linker, wherein the nucleic acid complex linker comprises a hairpin motif comprising a double stranded stem segment, a single stranded loop segment, a first end tail segment, and a second end tail segment; wherein the quencher and the first fluorescent molecule are configured to quench when the nucleic acid complex linker is in the form of a hairpin motif; wherein the quencher and the first fluorescent molecule are not configured to quench when the nucleic acid complex linker is in the form of a single stranded motif; wherein the fluorescent molecule is in a position and configured to move the position of the fluorescent molecule away from the quencher relative to the quencher when the CD19 antigen moves upon binding to a blinatumomab providing a detected light and/or fluorescent signal; comparing the detected light and/or fluorescent signal to normal or reference light and/or fluorescent signal indicative of a high risk of cytokine release syndrome, wherein if the detected light and/or fluorescent signal is the same or similar or higher than a normal or reference light and/or fluorescent signal it is indicative of a high risk of cytokine release syndrome.

In certain embodiments, the detected light and/or fluorescent signal is recorded on a computer readable medium providing a quantitative result.

In certain embodiments, methods further comprise comparing the detected light and/or fluorescent signal to normal or reference light and/or fluorescent signal.

In certain embodiments, methods further comprise comparing the detected light and/or fluorescent signal to normal or reference light and/or fluorescent signal which is done on a computer, providing a quantitative or comparative result.

In certain embodiments, methods further comprise communicating to the patient or a medical professional the quantitative result, the comparative result, or that the patent is at a high risk of cytokine release syndrome.

In certain embodiments, methods further comprise diagnosing the patient with a high risk of cytokine release syndrome if administered blinatumomab.

In certain embodiments, this disclosure relates to devices and methods for imaging transient and rare mechanical events in cancer cells or any non-immune or immune cell expressing endogenous proteins, receptors, or CARs. In certain embodiments, this disclosure contemplates devices which can measure the biomechanical forces of any cell including but not limited to cancer cells, endogenous non-immune/immune cells, or engineered immune cells expressing CARs (or other motifs which increases recognition of cancer cells). Furthermore, a device comprises a molecular beacon as a linker between a solid surface and an antigen, and a locking oligonucleotide that hybridizes to a portion of the hairpin stem loop and stem of the molecular beacon when the molecular beacon unravels or melts due to pulling forces on a complex of a bispecific T cell engager, the cancer antigen (e.g. CD19 but this could be any antigen expressed on cancer cells) and CD3 expressed on T-cells (however this technology could be targeted to any antigen expressed on immune or non-immune cells).

In certain embodiments, the bispecific engager specifically binds a cancer antigen such as cluster of differentiation 19 (CD19), cluster of differentiation 10 (CD10), cluster of differentiation 20 (CD20), cluster of differentiation 33 (CD33), cluster of differentiation 38 (CD38), CD70 (tumor necrosis factor ligand superfamily member 7), CD133 (prominin 1), CD171 (L1 cell adhesion molecule), (EGFR) epidermal growth factor receptor, (HER2) human epidermal growth factor receptor 2, EGFR vIII (epidermal growth factor receptor variant 3) (MUC1) mucin1, (MUC16) mucin16, (EpCAM) epithelial cell adhesion molecule, (AFP) alpha-fetoprotein, (FAP) familial adenomatous polyposis, (CEA) carcinoembryonic antigen, (PSCA) prostate stem cell antigen, (PSMA) prostate-specific membrane antigen, (PSA) prostate-specific antigen, (AXL) AXL receptor tyrosine kinase, (DLL3) delta-like 3, (EPHA2) EPH receptor A2, (FRα) folate receptor alpha, (LMP1) Epstein-Barr virus latent membrane protein 1, (MAGE) melanoma antigen gene protein, MAGE-A1, MAGE-A3, MAGE-A4, (DR5) death receptor 5, (NKG2D) natural killer group 2 member D receptor, (CAIX) carbonic anhydrase IX, (TAG-72) tumor-associated glycoprotein 72, (GUCY2C) guanylate cyclase 2C, (ANTXR1) anthrax toxin receptor 1, (GSPG4) general secretion pathway protein G, (ROR) RAR-related orphan receptors, ROR1 (receptor tyrosine kinase like orphan receptor 1), IL13RA2 (Interleukin 13 Receptor Subunit Alpha 2), Wilms' tumor 1 (WT1), Survivin, Tn (aGalNAc-O-Ser/Thr), sialyl-Tn (aNeuAc2,6-aGalNAc-O-Ser/Thr), TF (bGal1,3-aGalNAc-O-Ser/Thr), CA 19-9 (Neu5Acα2-3Galβ1-3 [Fucα1-4]GlcNAcβ), Telomerase reverse transcriptase (TERT), Beta-hCG (Human chorionic gonadotropin), p53, Ras, bladder tumor antigen (BTA), antibody specific antigen Om5, GD2 (Ganglioside GD2), integrin alpha-v/beta-6, mesothelin antigen, BCMA (TNF receptor superfamily member 17\B-cell maturation protein), CD123 (interleukin 3 receptor subunit α\CD123 antigen), CD138 (syndecan 1), CD22 (SIGLEC2), CD5 (lymphocyte antigen T1/Leu-1), Ig kappa chain, LeY (fucosyltransferase 3/Lewis Blood Group), NKG2D ligand (killer cell lectin like receptor K1/CD314), WT1 (Wilms' tumor antigen 1), C-Met (MET proto-oncogene), CAIX (carbonic anhydrase 9), GPC3 (glypican 3), HPV16-F6 (human papillomavirus F6 protein), MARTI (melan-A), NY-ESO-1 (cancer/testis antigen 1B), PD-L1 (CD274 molecule), PSMA (folate hydrolase 1), or VEGFR2 (kinase insert domain receptor/vascular endothelial growth factor receptor 2).

In certain embodiments, the cancer is a solid tumor, cellular malignancy, or hematological malignancy such as leukemia, lymphoma, or multiple myeloma. In certain embodiments, the cancer is ependymoma, lung cancer, non-small cell lung cancer, small cell lung cancer, bronchus cancer, mesothelioma, malignant pleural mesothelioma, lung adenocarcinoma, breast cancer, prostate cancer, colon cancer, rectum cancer, colorectal cancer, gastrointestinal cancer, stomach cancer, esophageal cancer, ovarian cancer, cervical cancer, melanoma, kidney cancer, pancreatic cancer, pancreatic ductal adenocarcinoma (PDA), thyroid cancer, brain cancer, glioblastoma (GBM), medulloblastoma, glioma, neuroblastoma, liver cancer, bladder cancer, uterine cancer, bone cancer, osteosarcoma, sarcoma, rhabdomyosarcoma, Ewing's sarcoma, retinoblastoma, nasopharyngeal carcinoma.

In Fluorescence Resonance Energy Transfer (FRET), energy of an excited fluorophore (sometime referred to as a “donor”) is transferred to a neighboring acceptor molecule, e.g., limiting fluorescence of the donor fluorophore due to quenching (“quencher”). The distance between the donor and the quencher is related to the intensity of quenching. When donor fluorophore and quencher are separated, quenching is reduced or eliminated, and fluorescence is intensified. Likewise, when the donor and quencher are close, quenching is typically efficient, e.g., low intensity or dark. The range over which quenching occurs is unique for each fluorophore donor and quencher pair. The quencher molecule can be another fluorescent dye or a non-fluorescent dark quencher. See Johansson, Choosing Reporter-Quencher Pairs for Efficient Quenching Through Formation of Intramolecular Dimers, Methods in Molecular Biology, 2006, 335:17-29. Chapter 2 Fluorescent Energy Transfer Nucleic Acid Probes: Designs and Protocols.

Examples of fluorescent dyes include fluorescein dyes, fluorescein isothiocyanate (FITC) dyes, rhodamine-based dyes such as tetramethylrhodamine (TMR) dyes, carboxytetramethylrhodamine dyes (TAMRA), 6-(2-carboxyphenyl)-1,11-diethyl-3,4,8,9,10,11-hexahydro-2H-pyrano[3,2-g: 5,6-g′]diquinolin-1-ium dyes (ATTO™ dyes) and other such as 4,4-difluoro-4-bora-3a,4a-diaza-s-indacene (BODIPY) dyes, BODIPY FL™, Oregon Green 488™, Rhodamine Green™, Oregon Green 514™, TET™, Cal Gold™, BODIPY R6G™, Yakima Yellow™, JOF™, HEX™, Cal Orange™, BODIPY TMR-X™, Quasar-570/Cy3™, Rhodamine Red-X™, Redmond Red™, BODIPY 581/591™, ROX™, Cal Red/Texas Red™, BODIPY TR-X™, BODIPY 630/665-X™, Pulsar-650™, cyanine dyes, Cy3B™, Quasar-670/Cy5™, Cy3.5™, and Cy5.5™. Examples of quenchers include fluorescein, rhodamine, and cyanine dyes, dabcyl, and Black Hole Quenchers™ (BHQs). Dark quenchers include dabcyl, QSY 35™, BHQ-0 ™, Eclipse™, BHQ-1 ™, QSY 7 ™, QSY 9 ™, BHQ-2 ™, ElleQuencher™, Iowa Black™, QSY 21™, BHQ-3™.

In certain embodiments the locking oligonucleotide comprises a toehold segment. In certain embodiments, this disclosure relates to methods of locking, unlocking, and imaging cellular events using labeled locking oligonucleotides or toehold oligonucleotides disclosed herein.

In certain embodiments, this disclosure contemplates a system comprising: a) a device comprising: i) a cell, cancer cell, or an immune cell, a bispecific engager, that specifically binds a cell antigen and another antigen; ii) a nucleic acid complex linker having a first end and a second end, wherein the nucleic acid complex linker is linked to the antigen at the first end; iii) a surface connected to the nucleic acid complex linker at the second end; wherein the nucleic acid complex linker comprises a hairpin motif comprising a double stranded stem segment, a single stranded loop segment, a first end tail segment, and a second end tail segment; and b) a locking oligonucleotide that hybridizes with the double stranded stem segment and the single stranded loop segment when a bispecific T cell engager binds the cancer antigen and other antigen and unravels or melts the hairpin motif providing an extended form derived from a single stranded motif. In certain embodiments, fluorescent and quencher pairs are arranged to provide a signal when extended.

In certain embodiments, this disclosure contemplates a system comprising: a) a device comprising: i) a bispecific engager and an antigen ii) a nucleic acid complex linker having a first end and a second end, wherein the nucleic acid complex linker is linked to the antigen at the first end; iii) a surface connected to the nucleic acid complex linker at the second end; iv) a quencher conjugated to the nucleic acid complex linker wherein the quencher position remains static when the antigen moves; and v) a first fluorescent molecule conjugated to the nucleic acid complex linker wherein the fluorescent molecule is configured to move its position relative to the quencher when the antigen moves; wherein the nucleic acid complex linker comprises a hairpin motif comprising a double stranded stem segment, a single stranded loop segment, a first end tail segment, and a second end tail segment; wherein the quencher and the first fluorescent molecule are configured to quench when the nucleic acid complex linker is in the form of a hairpin motif; and wherein the quencher and the first fluorescent molecule are not configured to quench when the nucleic acid complex linker is in the form of a single stranded motif; and b) a locking oligonucleotide that hybridizes with the double stranded stem segment and the single stranded loop segment.

In certain embodiments, the locking oligonucleotide comprises a label such as a fluorescent molecule when a receptor binds the ligand and unravels the hairpin motif providing an extended form derived from a single stranded motif.

In certain embodiments, the disclosure contemplates a nucleic acid complex linker configured such that it only binds a locking oligonucleotide when it is mechanically denatured. In certain embodiments, the nucleic acid complex linker is designed with a hidden (cryptic) binding segment to the locking oligonucleotide, i.e., locking oligonucleotide does not bind to the nucleic acid complex linker during static conditions; however, when the ligand moves, then the cryptic site is exposed, thus permitting the locking oligonucleotide to bind with the cryptic binding segment. The nucleic acid complex is configured to have mechanical selectively of at least or greater than 1:10 and in some cases this is 1:100 and 1:1000 or greater. Mechanical selectively is the ratio of the locking oligonucleotide binding to the cryptic segment with no ligand movement compared the locking oligonucleotide binding to the cryptic segment once the ligand moves and the nucleic acid complex linker melts due to forces.

In certain embodiments, this disclosure contemplates methods of detecting or imaging a tension force using a bispecific engager to facilitate a pulling force on a cancer antigen and another antigen comprising a) contacting i) a bispecific engager and the cancer antigen and the other antigen or cells containing the same; ii) a device comprising, a nucleic acid complex linker having a first end and a second end, wherein the nucleic acid complex linker is linked to the antigen at the first end, and a surface connected to the nucleic acid complex linker at the second end; wherein the nucleic acid complex linker comprises a hairpin motif comprising a double stranded stem segment, a single stranded loop segment, a first end tail segment, and a second end tail segment; and iii) a locking oligonucleotide optionally comprising a label that hybridizes with the double stranded stem segment and the single stranded loop segment, when a receptor binds the cancer antigen, unraveling the hairpin motif providing an extended form of a single stranded motif; and b) detecting the label on the locking oligonucleotide. In certain embodiments, the label is a fluorescent molecule and detecting is observing the fluorescence of the label. In certain embodiments, the fluorescence is used to generate an image.

In any embodiments disclosed herein, a quencher and a fluorescent molecule may be in reverse or in opposite positions, i.e., a quencher may optionally be a fluorescent molecule and a fluorescent molecule may be a quencher. In any embodiments disclosed herein, the quencher may be absent, or the quencher may optionally be a fluorescent molecule, optionally of different excitation maximums and/or emission maximums in the case that two or more fluorescent molecules are used in the device or system. In certain embodiments, the excitation maximums and/or emission maximums differ by more than 50 nm, 100 nm, 150 nm, or 200 nm and optionally the excitation maximums and/or emission maximums differ by less than 150 nm, 200 nm, or 400 nm. In certain embodiments, the nucleic acid complex linker does not contain a fluorescent molecule or quencher, or neither a fluorescent molecule nor a quencher.

In certain embodiments, devices further comprise a locking oligonucleotide or toehold oligonucleotide comprising a sequence with only one nucleotide that base pairs with the last nucleotide of the double stranded stem segment, adjacent to the single stranded loop, followed by the reverse complement of the single stranded loop segment followed by the reverse complement of the double stranded stem segment. In certain embodiments, the sequence with only one nucleotide that base pairs with the last nucleotide of the double stranded stem segment followed by the reverse complement of the single stranded loop segment followed by the reverse complement of the double stranded stem segment is between 16 and 18 nucleotides, or 15 and 19 nucleotide, or 15 and 20 nucleotides.

In certain embodiments, the locking oligonucleotide or toehold oligonucleotide is conjugated to a second fluorescent molecule wherein the first fluorescent molecule and second fluorescent molecule have different excitation maximums and/or emission maximums.

In certain embodiments, this disclosure relates to methods of detecting a light and/or fluorescent signal from a cell binding a bispecific engager comprising the steps of: a) exposing a device disclosed herein to a cell containing a cancer antigen to the bispecific engager, antibody, or any protein or moiety recognized by the cancer cell under conditions such that the device presents to the cancer cells a DNA-based probe which ligates to the cancer cell using any of the means described above; and b) detecting the light and/or fluorescent signal. In certain embodiments, the light and/or fluorescent signals are used to create an image.

In certain embodiments, it is contemplated that devices disclosed herein are used to measure biomechanical forces for any cells endogenous, engineered, or otherwise. This technology is not limited to cancer. Measuring biomechanical forces from any cell (immune, cancer, or otherwise) could be useful in pathological settings (cancer, autoimmunity, hypertension, neurological disorders, etc.).

In certain embodiments, this disclosure relates to methods of preventing cytokine release syndrome comprising obtaining a sample of T cells or other immune cells from a patient diagnosed with cancer; contacting the T cells with blinatumomab and to a device comprising: a nucleic acid complex linker having a first end and a second end, wherein the nucleic acid complex linker is linked to CD19 antigen at the first end; a surface connected to the nucleic acid complex linker at the second end; a quencher conjugated to the nucleic acid complex linker; and a fluorescent molecule conjugated to the nucleic acid complex linker, wherein the nucleic acid complex linker comprises a hairpin motif comprising a double stranded stem segment, a single stranded loop segment, a first end tail segment, and a second end tail segment; wherein the quencher and the first fluorescent molecule are configured to quench when the nucleic acid complex linker is in the form of a hairpin motif; wherein the quencher and the first fluorescent molecule are not configured to quench when the nucleic acid complex linker is in the form of a single stranded motif; wherein the fluorescent molecule is in a position and configured to move the position of the fluorescent molecule away from the quencher relative to the quencher when the CD19 antigen moves upon binding to a blinatumomab providing a detected light and/or fluorescent signal; comparing the detected light and/or fluorescent signal to normal or reference light and/or fluorescent signal indicative of a high risk of cytokine release syndrome, wherein if the detected light and/or fluorescent signal is the same or similar or higher than a normal or reference light and/or fluorescent signal it is indicative of a high risk of cytokine release syndrome.

In certain embodiments, the detected light and/or fluorescent signal is recorded on a computer readable medium providing a quantitative result.

In certain embodiments, methods further comprise comparing the detected light and/or fluorescent signal to normal or reference light and/or fluorescent signal.

In certain embodiments, methods further comprise comparing the detected light and/or fluorescent signal to normal or reference light and/or fluorescent signal which is done on a computer providing a comparative result.

In certain embodiments, methods further comprise communicating to the patient or a medical professional the quantitative result, the comparative result, or that the patent is at a high risk of cytokine release syndrome.

In certain embodiments, methods further comprise diagnosing the patient with a high risk of cytokine release syndrome if administered blinatumomab.

In certain embodiments, this disclosure relates to methods of detecting a light and/or fluorescent signal from a blood sample of a subject comprising a cell expressing a CD3 antigen the steps of: a) exposing a device to a cell comprising CD3 antigen and a bi-specific T-cell engager that specifically binds a CD3 antigen and a CD19 antigen in the presence of a locking oligonucleotide; wherein the device comprises: i) a CD19 antigen; ii) a nucleic acid complex linker having a first end and a second end, wherein the nucleic acid complex linker is linked to the CD19 antigen at the first end; iii) a surface connected to the nucleic acid complex linker at the second end; iv) a quencher conjugated to the nucleic acid complex linker; and v) a first fluorescent molecule conjugated to the nucleic acid complex linker wherein the fluorescent molecule is configured to move its position relative to the quencher when the CD19 antigen moves upon binding to the bi-specific T-cell engager and CD3; wherein the nucleic acid complex linker comprises a hairpin motif comprising a double stranded stem segment, a single stranded loop segment, a first end tail segment, and a second end tail segment; wherein the quencher and the first fluorescent molecule are configured to quench when the nucleic acid complex linker is in the form of a hairpin motif; wherein the quencher and the first fluorescent molecule are not configured to quench when the nucleic acid complex linker is in the form of a single stranded motif; and wherein the bi-specific T-cell engager binds and pulls the CD19 antigen away from the surface to unravel the hairpin motif into the single stranded motif removing the first fluorescent molecule from proximity to the quencher producing a light and/or fluorescent signal and the locking oligonucleotide hybridizes to the single stranded motif under conditions such that the nucleic acid complex linker is locked in an extended form derived from the single stranded motif; and b) detecting the light and/or fluorescent signal.

In certain embodiments, the subject is receiving a chemotherapy treatment including a bi-specific T-cell engager that specifically binds both CD3 and CD19.

In certain embodiments, the bi-specific T-cell engager is blinatumomab.

In certain embodiments, the detected light and/or fluorescent signal indicates an increased risk that the subject may not be responsive to the chemotherapy treatment or that the chemotherapy treatment has stopped working.

In certain embodiments, the detected light and/or fluorescent signal indicates an increased risk that the subject is experiencing cytokine release syndrome.

In certain embodiments, the sample is from subject that is not yet received a chemotherapy treatment including a bi-specific T-cell engager that specifically binds both CD3 and CD19 and is be considered for such treatment.

In certain embodiments, the locking oligonucleotide comprises a sequence that is only one nucleotide that base pairs with the last nucleotide of the double stranded stem segment followed by the reverse complement of the single stranded loop segment followed by the reverse complement of the double stranded stem segment.

In certain embodiments, this disclosure relates to devices comprising nucleotide polymers comprising SEQ ID NO: 2, SEQ ID NO: 5; SEQ ID NO: 6 (without locking nucleic acid) or SEQ ID NO: 2, SEQ ID NO: 5; SEQ ID NO: 6, and SEQ ID NO: 7 (with locking nucleic acid).

In certain embodiments, this disclosure relates to devices comprising nucleotide polymers comprising SEQ ID NO: 3, SEQ ID NO: 5; SEQ ID NO: 6 (without locking nucleic acid) or SEQ ID NO: 3, SEQ ID NO: 5; SEQ ID NO: 6, and SEQ ID NO: 7 (with locking nucleic acid).

In certain embodiments, this disclosure relates to devices comprising nucleotide polymers comprising SEQ ID NO: 4, SEQ ID NO: 5; SEQ ID NO: 6 (without locking nucleic acid) or SEQ ID NO: 4, SEQ ID NO: 5; SEQ ID NO: 6, and SEQ ID NO: 7 (with locking nucleic acid).

In certain embodiments, this disclosure relates to devices comprising: i) a CD19 antigen; ii) a nucleic acid complex linker having a first end and a second end, wherein the nucleic acid complex linker is linked to the CD19 antigen at the first end; iii) a surface connected to the nucleic acid complex linker at the second end; iv) a quencher conjugated to the nucleic acid complex linker; and v) a first fluorescent molecule conjugated to the nucleic acid complex linker wherein the fluorescent molecule is configured to move its position relative to the quencher when the CD19 antigen moves upon binding to a receptor of the CD19 antigen such as a bi-specific T-cell engager; wherein the nucleic acid complex linker comprises a hairpin motif comprising a double stranded stem segment, a single stranded loop segment, a first end tail segment, and a second end tail segment; wherein the quencher and the first fluorescent molecule are configured to quench when the nucleic acid complex linker is in the form of a hairpin motif; and wherein the quencher and the first fluorescent molecule are not configured to quench when the nucleic acid complex linker is in the form of a single stranded motif.

In certain embodiments, this disclosure relates to methods of detecting a light and/or fluorescent signal from a blood sample of a subject comprising a cell expressing a CD3 antigen or other immune cell antigen comprising the steps of: a) exposing a device to a cell comprising CD3 antigen and a bi-specific T-cell engager that specifically binds a CD3 antigen and a second antigen in the presence of a locking oligonucleotide; wherein the device comprises: i) a second cancer antigen; ii) a nucleic acid complex linker having a first end and a second end, wherein the nucleic acid complex linker is linked to the second antigen at the first end; iii) a surface connected to the nucleic acid complex linker at the second end; iv) a quencher conjugated to the nucleic acid complex linker; and v) a first fluorescent molecule conjugated to the nucleic acid complex linker wherein the fluorescent molecule is configured to move its position relative to the quencher when the second antigen moves upon binding to the bi-specific T-cell engager, bound to CD3 or other immune cell antigen on the device; wherein the nucleic acid complex linker comprises a hairpin motif comprising a double stranded stem segment, a single stranded loop segment, a first end tail segment, and a second end tail segment; wherein the quencher and the first fluorescent molecule are configured to quench when the nucleic acid complex linker is in the form of a hairpin motif; wherein the quencher and the first fluorescent molecule are not configured to quench when the nucleic acid complex linker is in the form of a single stranded motif; and wherein the bi-specific T-cell engager binds CD3 or other immune cell antigen and the second cancer antigen and pulls the second antigen away from the surface to unravel the hairpin motif into the single stranded motif removing the first fluorescent molecule from proximity to the quencher producing a light and/or fluorescent signal and the locking oligonucleotide hybridizes to the single stranded motif under conditions such that the nucleic acid complex linker is locked in an extended form derived from the single stranded motif; and b) detecting the light and/or fluorescent signal.

In certain embodiment, the subject is receiving a chemotherapy treatment including a bi-specific T-cell engager that specifically binds both CD3 or other immune cell antigen and the second cancer antigen. In certain embodiment, the bi-specific T-cell engager is blinatumomab.

In certain embodiment, the detected light and/or fluorescent signal indicates an increased risk that the subject is not responsive to the chemotherapy treatment or that the chemotherapy treatment has stopped working.

In certain embodiment, the detected light and/or fluorescent signal indicates an increased risk that the subject is experiencing cytokine release syndrome.

In certain embodiment, the sample is from subject that is not yet received a chemotherapy treatment including a bi-specific T-cell engager, then is to be considered for such treatment.

In certain embodiment, the locking oligonucleotide comprises a sequence that is only one nucleotide that base pairs with the last nucleotide of the double stranded stem segment followed by the reverse complement of the single stranded loop segment followed by the reverse complement of the double stranded stem segment.

In certain embodiment, the second antigen is cluster of differentiation 19 (CD19), cluster of differentiation 10 (CD10), cluster of differentiation 20 (CD20), cluster of differentiation 33 (CD33), cluster of differentiation 38 (CD38), CD70 (tumor necrosis factor ligand superfamily member 7), CD133 (prominin 1), CD171 (L1 cell adhesion molecule), (EGFR) epidermal growth factor receptor, (HER2) human epidermal growth factor receptor 2, EGFR vIII (epidermal growth factor receptor variant 3) (MUC1) mucin1, (MUC16) mucin16, (EpCAM) epithelial cell adhesion molecule, (AFP) alpha-fetoprotein, (FAP) familial adenomatous polyposis, (CEA) carcinoembryonic antigen, (PSCA) prostate stem cell antigen, (PSMA) prostate-specific membrane antigen, (PSA) prostate-specific antigen, (AXL) AXL receptor tyrosine kinase, (DLL3) delta-like 3, (EPHA2) EPH receptor A2, (FRα) folate receptor alpha, (LMP1) Epstein-Barr virus latent membrane protein 1, (MAGE) melanoma antigen gene protein, MAGE-A1, MAGE-A3, MAGE-A4, (DR5) death receptor 5, (NKG2D) natural killer group 2 member D receptor, (CAIX) carbonic anhydrase IX, (TAG-72) tumor-associated glycoprotein 72, (GUCY2C) guanylate cyclase 2C, (ANTXR1) anthrax toxin receptor 1, (GSPG4) general secretion pathway protein G, (ROR) RAR-related orphan receptors, ROR1 (receptor tyrosine kinase like orphan receptor 1), IL13RA2 (Interleukin 13 Receptor Subunit Alpha 2), Wilms' tumor 1 (WT1), Survivin, Tn (aGalNAc-O-Ser/Thr), sialyl-Tn (aNeuAc2,6-aGalNAc-O-Ser/Thr), TF (bGal1,3-aGalNAc-O-Ser/Thr), CA 19-9 (Neu5Acα2-3Galβ1-3 [Fucα1-4]GlcNAcβ), Telomerase reverse transcriptase (TERT), Beta-hCG (Human chorionic gonadotropin), p53, Ras, bladder tumor antigen (BTA), antibody specific antigen Om5, GD2 (Ganglioside GD2), integrin alpha-v/beta-6, or mesothelin antigen, BCMA (TNF receptor superfamily member 17\B-cell maturation protein), CD123 (interleukin 3 receptor subunit α\CD123 antigen), CD138 (syndecan 1), CD22 (SIGLEC2), CD5 (lymphocyte antigen T1/Leu-1), Ig kappa chain, LeY (fucosyltransferase 3/Lewis Blood Group), NKG2D ligand (killer cell lectin like receptor K1/CD314), WT1 (Wilms' tumor antigen 1), C-Met (MET proto-oncogene), CAIX (carbonic anhydrase 9), GPC3 (glypican 3), HPV16-E6 (human papillomavirus E6 protein), MARTI (melan-A), NY-ESO-1 (cancer/testis antigen 1B), PD-L1 (CD274 molecule), PSMA (folate hydrolase 1), or VEGFR2 (kinase insert domain receptor/vascular endothelial growth factor receptor 2).

In certain embodiment, this disclosure relates to devices comprises: i) an antigen found on cancer cells, and/or immune cells, and/or non-immune cells; ii) a nucleic acid complex linker having a first end and a second end, wherein the nucleic acid complex linker is linked to the antigen at the first end; iii) a surface connected to the nucleic acid complex linker at the second end; iv) a quencher conjugated to the nucleic acid complex linker; and v) a first fluorescent molecule conjugated to the nucleic acid complex linker wherein the fluorescent molecule is configured to move its position relative to the quencher when the antigen moves upon binding to a receptor of the antigen; wherein the nucleic acid complex linker comprises a hairpin motif comprising a double stranded stem segment, a single stranded loop segment, a first end tail segment, and a second end tail segment; wherein the quencher and the first fluorescent molecule are configured to quench when the nucleic acid complex linker is in the form of a hairpin motif; and wherein the quencher and the first fluorescent molecule are not configured to quench when the nucleic acid complex linker is in the form of a single stranded motif.

In certain embodiments, the antigen is a bispecific engager that binds CD3 or other immune cell antigen and an second antigen selected from cluster of differentiation 19 (CD19), cluster of differentiation 10 (CD10), cluster of differentiation 20 (CD20), cluster of differentiation 33 (CD33), cluster of differentiation 38 (CD38), CD70 (tumor necrosis factor ligand superfamily member 7), CD133 (prominin 1), CD171 (L1 cell adhesion molecule), (EGFR) epidermal growth factor receptor, (HER2) human epidermal growth factor receptor 2, EGFR vIII (epidermal growth factor receptor variant 3) (MUC1) mucin1, (MUC16) mucin16, (EpCAM) epithelial cell adhesion molecule, (AFP) alpha-fetoprotein, (FAP) familial adenomatous polyposis, (CEA) carcinoembryonic antigen, (PSCA) prostate stem cell antigen, (PSMA) prostate-specific membrane antigen, (PSA) prostate-specific antigen, (AXL) AXL receptor tyrosine kinase, (DLL3) delta-like 3, (EPHA2) EPH receptor A2, (FRα) folate receptor alpha, (LMP1) Epstein-Barr virus latent membrane protein 1, (MAGE) melanoma antigen gene protein, MAGE-A1, MAGE-A3, MAGE-A4, (DR5) death receptor 5, (NKG2D) natural killer group 2 member D receptor, (CAIX) carbonic anhydrase IX, (TAG-72) tumor-associated glycoprotein 72, (GUCY2C) guanylate cyclase 2C, (ANTXR1) anthrax toxin receptor 1, (GSPG4) general secretion pathway protein G, (ROR) RAR-related orphan receptors, ROR1 (receptor tyrosine kinase like orphan receptor 1), IL13RA2 (Interleukin 13 Receptor Subunit Alpha 2), Wilms' tumor 1 (WT1), Survivin, Tn (aGalNAc-O-Ser/Thr), sialyl-Tn (aNeuAc2,6-aGalNAc-O-Ser/Thr), TF (bGal1,3-aGalNAc-O-Ser/Thr), CA 19-9 (Neu5Acα2-3Galβ1-3 [Fucα1-4]GlcNAcβ), Telomerase reverse transcriptase (TERT), Beta-hCG (Human chorionic gonadotropin), p53, Ras, bladder tumor antigen (BTA), antibody specific antigen Om5, GD2 (Ganglioside GD2), integrin alpha-v/beta-6, or mesothelin antigen, BCMA (TNF receptor superfamily member 17\B-cell maturation protein), CD123 (interleukin 3 receptor subunit α\CD123 antigen), CD138 (syndecan 1), CD22 (SIGLEC2), CD5 (lymphocyte antigen T1/Leu-1), Ig kappa chain, LeY (fucosyltransferase 3/Lewis Blood Group), NKG2D ligand (killer cell lectin like receptor K1/CD314), WT1 (Wilms' tumor antigen 1), C-Met (MET proto-oncogene), CAIX (carbonic anhydrase 9), GPC3 (glypican 3), HPV16-E6 (human papillomavirus E6 protein), MARTI (melan-A), NY-ESO-1 (cancer/testis antigen 1B), PD-L1 (CD274 molecule), PSMA (folate hydrolase 1), or VEGFR2 (kinase insert domain receptor/vascular endothelial growth factor receptor 2).

### Microenvironmental Factors Regulate T-Cell Activation by Tuning T-Cell Receptor Biomechanical Properties

Obesity is a major public health concern. Obesity is also associated with an increased risk for cancer. Patients with obesity tend to have an increased tumor burden and decreased T-cell function. Lydia Dyck et al. report suppressive effects of the obese tumor microenvironment on CD8 T cell infiltration and effector function. J Exp Med. 2022 Mar. 7; 219 (3): e20210042. It remains unclear how obesity compromises T-cell mediated immunity. Adipocytes are specialized cells for the storage of fat. The adipocyte niche was modeled using the secretomes released from adipocytes and their precursor stromal cells to investigate how these factors modulated T cell function. The secretome altered antigen-specific T cell receptor (TCR) triggering and activation. RNA-sequencing analysis identified hundreds of gene targets modulated by the secretome including those associated with cytoskeleton regulation and actin polymerization. Molecular force probes were used in experiments to show that the adipocyte niche dampens force transmission to the TCR-pMHC complex and conversely, bone marrow stromal cell secreted factors significantly strengthen TCR forces. Importantly, secretome-mediated TCR force modulation mirrored the changes in T cell functional response in human T cells using the clinically approved immunotherapy, blinatumomab. Thus, these experiments indicate that the adipocyte niche contributes to T cell dysfunction by modulating its cytoskeleton and dampening TCR activation by reducing TCR forces in agreement with the mechanosensor model of T cell activation.

Obesity is reported to have suppressive effects on cancer immunotherapies. Experiments were performed to understand the impacts immunotherapies have in targeting hematological malignancies such as when using chimeric antigen receptor (CAR) T cells and bispecific T-cell engagers (BiTEs), which are clinically approved treatments for patients with relapsed or refractory B-cell acute lymphoblastic leukemia (B-ALL), diffuse large B-cell lymphoma (DLBCL), multiple myeloma, and mantle cell lymphoma.

Experiments reported herein indicate that the secretomes of bone marrow stromal cells and adipocytes differentially impact T-cell function and the efficacy of immunotherapies. Stromal cell-conditioned media (SCM) and adipocyte-conditioned media (ACM) differentially alter T-cell activation. Functionally, SCM increased cytokine production (IFN-γ and TNF-α), and the expression of cytolytic mediators (perforin and GranzymeB). In contrast, ACM suppressed each of these parameters in T cells as well as TCR signal transduction (upregulation of Nur77) and the surface expression of T-cell activation markers (CD25 and CD69). RNA sequencing experiments performed on T-cells cultured in the secretomes of bone marrow stromal cells and adipocytes revealed rapid and distinctive gene expression changes in naive T-cells. Notably, SCM induced high expression of critical adaptors or mediators that link actin polymerization to TCR signal transduction, specifically Lcp2/SLP-7631. In contrast, ACM-mediated suppression of T-cell function occurred rapidly at the gene expression level in naive T-cells with genes encoding TCR machinery and actin polymerization being significantly suppressed relative to levels observed in T cells cultured in unconditioned media or SCM.

Transcriptional differences in cytoskeletal genes were validated by showing that SCM and ACM differentially alter total and filamentous actin (F-actin) levels in the T-cell cytoskeleton at the protein level, with SCM leading to increased actin levels and ACM leading to actin suppression. DNA-based molecular force sensors were employed to measure TCR forces in cells treated with conditioned media. Soluble factors in the microenvironment dictated TCR mechanical forces during initial antigen recognition, which demonstrates an association between microenvironment-mediated alterations in cytoskeletal organization and the biomechanics of the TCR. The bispecific T cell engager (BiTE), blinatumomab is a clinically approved immunotherapy for B-cell acute lymphoblastic leukemia (B-ALL). Experiments reported herein indicate that biomechanical forces transmitted through blinatumomab target cell killing in co-culture assays of human T-cells with human B-ALL cells.

Using methods and devices disclosed herein one can measure changes in actin polymerization and forces directly exerted by the TCR, as well as T-cell activation markers and effector functions, after exposure to complex bone marrow stromal cell and adipocyte secretomes. Additionally, a set of cytoskeletal genes were uncovered that are significantly downregulated upon ACM treatment which may be responsible for the changes in TCR mechanics. These genes include Mylk3, Itga4, Actn1, Pik3r2, Itga7, Arghef4, Lpar5, Pip4k2b, Pfn2, Iqgap3, Itga1, Pdgfb, and Itga5, and play a role in actin polymerization, stress fiber formation, actomyosin assembly and contraction, focal adhesion formation, and adherents junction formation. Additionally, Wasf1 (also known as WASP-family verprolin homologous protein 1 (WAVE1)) is uniquely upregulated in T-cells exposed to the bone marrow stromal cell secretome, providing a potential mechanism for the increased actin polymerization and TCR mechanics seen in these cells. Taken together, these results demonstrate that the actin cytoskeleton and endogenous TCR forces are affected by adipocytic and stromal cell microenvironments. Furthermore, adipocyte-secreted factors may hinder T-cell function by interfering with cytoskeletal mechanics.

T-cell mechanical forces play an important role in cancer cell killing. Cancer cells with high metastatic potential tend to be softer than cancer cells with a low metastatic potential, which provides a mechanism for immune evasion because T-cells cannot accumulate F-actin at the immunological synapse and exert strong mechanical forces on mechanically soft cells. The soluble microenvironment can alter T-cell biomechanics in addition to F-actin levels, immunological synapse formation, and cancer cell killing. When mechanical forces are reduced, T-cells lost most of their effector functions including cytokine and cytolytic mediator production, TCR triggering, immunological synapse formation, and cytotoxicity.

The blinatumomab harnesses endogenous T-cell function by bridging CD3+ T-cells and CD19+ B-cells to promote immunological synapse formation and cytotoxicity. Using DNA-based tension probes, T-cells were shown to exert a quantifiable mechanical force on blinatumomab. Additionally, the soluble microenvironment modulates mechanical forces exerted by T-cells on blinatumomab. Decreased mechanical forces through blinatumomab are correlated with poor immunological synapse formation and low levels of cytotoxicity, providing a biomarker associated with the function of the drug.

One can use a biomechanical readout, or “biomechanical biomarkers,” as a method to perform quality control on cell-based immunotherapies. Using methods and devices disclosed herein one can test any ligand of interest in a 96-well plate format and obtain reports providing a correlation between TCR forces and T-cell function. Leveraging T cell biomechanics as an additional quality control marker for cell-based immunotherapies can complement current strategies to lead to more successful immunotherapy treatments.

### The Soluble Microenvironment Modulates Antigen-Specific T Cell Activation

As a model of T cell activation, transgenic OT1 mouse T-cells that specifically recognize the ovalbumin peptide (SIINFEKL, SEQ ID NO: 1) were utilized. To mimic different microenvironments, T-cells were exposed to harvested conditioned media from bone marrow stromal cells and differentiated adipocytes.

The procedure for generating conditioned media were done as reported in Wolins et al. J Lipid Res 47, 450-460 (2006) and Lee et al. Nat Commun 13, 1157 (2022). Adipocyte-secreted factors contain high levels of pro-inflammatory cytokines and chemokines including TNF-α and IL-6, which are indicators of chronic inflammation that is found in obesity. Soluble factors secreted by stromal cells enhance the immune response. OT-1 T-cells were cultured with OVA-peptide loaded splenocytes in unconditioned medium (UCM), stromal cell-conditioned medium (SCM) or adipocyte-conditioned medium (ACM) for 3 days before assessing T-cell effector functions. Relative to responses observed in UCM, OT-1 T-cells stimulated in ACM exhibited a significant decrease in IFN-γ, TNF-α, perforin, and granzyme B production by 49%, 61%, 52%, and 49%, respectively. In contrast, with the exception of TNF-α and IFN-γ, T-cells stimulated in SCM significantly increased their production of cytolytic mediators by 61% (perforin), and 27% (GzmB). Overall, these data demonstrate that the secretome of bone marrow stromal cells augments T-cell function; whereas the adipocyte secretome is suppressive to antigen-specific T-cells.

Experiments were performed to determine whether the conditioned media also modulated TCR signaling at earlier time points. The impact of UCM, SCM, and ACM on the phenotype of naive OT-1 T-cells were assessed with a focus on the surface expression of the TCR and CD8 coreceptor. To this end, OT-1 T cells were cultured for 24 hours in UCM, SCM or ACM (without stimulation). Naive T-cells cultured in SCM exhibited a decrease in their size and granularity, whereas T-cells cultured in ACM increased their size and granularity even in the absence of TCR stimulation. In addition to ACM-induced phenotypic changes, naive CD8+ T-cells cultured in the adipocyte secretome also appeared to develop sub-populations with unique scattering profiles, despite being clonal, antigen-specific T-cells.

Despite notable phenotypic changes in these conditions, T-cells did not exhibit significant alterations in their surface expression of the TCR-a subunit of the TCR nor the CD8a co-receptor, although a trend towards increased surface expression was noted in ACM. Experiments were performed to determine whether, TCR signaling was differentially impacted by the bone marrow stromal cell and adipocyte secretomes. In these experiments, Nur77-GFP expressing OT-1 T-cells were used because this transcription factor is an immediate early response which occurs within hours of TCR stimulation.

When naive OT-1 T-cells were cultured in UCM, SCM, or ACM without stimulation for 24 h, significant antigen-independent increases in homeostatic protein levels of Nur77 in naive T-cells cultured in SCM were observed. However, protein levels were similar in naive T-cells cultured in UCM and ACM. Next, T-cells were treated for 24 hours with UCM, SCM, or ACM prior to a 2-hour stimulation with OVA peptide-pulsed dendritic cells. After the 2-hour incubation period, the intracellular upregulation of Nur77-GFP was measured, as well as the surface expression of CD69 and CD25 given that these receptors are rapidly upregulated within a few hours of TCR stimulation. Relative to UCM, T-cells treated with ACM had a 26% decrease in the number of CD25^{HI} CD69^{HI} cells as well as a 59% decrease in the number of CD25^{HI} Nur77-GFP^{HI} cells; whereas similar responses in these markers of TCR stimulation were observed in T-cells cultured in UCM compared to SCM. In all, these data indicate that the bone marrow stromal cell and adipocyte secretomes differentially tune the effector capacity of naive T-cells prior to antigenic exposure by modulating TCR signaling properties.

### The Soluble Microenvironment Alters the Transcriptome of Naive CD3+ T Cells.

To gain a comprehensive understanding of how bone marrow stromal cells and adipocyte secretomes differentially prime naive T-cells, RNA-sequencing analysis was performed on naive murine CD3+ polyclonal T-cells after a 4-hour culture in UCM, SCM, or ACM. Principal component analysis (PCA) confirmed strong reproducibility across replicates and revealed that early transcriptional changes in naive CD8+ T-cells were largely influenced by the soluble microenvironment, with ACM-induced changes showing the largest divergence relative to gene expression probes observed in T cells cultured in UCM or SCM. In addition to inducing the greatest transcriptional changes, ACM induced a greater number of genes in naive T-cells relative to those observed in T-cells cultured in SCM. To this point, 450 genes were significantly upregulated, and 698 genes were significantly downregulated in naive T-cells cultured in ACM relative to UCM; whereas 97 genes were upregulated and 79 genes were downregulated in native T-cells cultured in SCM relative to UCM. Of these, 32.8% (415 genes) were uniquely upregulated in native T-cells cultured in ACM relative to UCM and 53.3% (675 genes) were downregulated only upon exposure to the adipocyte secretome. In contrast, 4.8% (61 genes) and 4.5% (57 genes) were uniquely up- and down-regulated, respectively, in naive T-cells cultured in SCM relative to ACM.

Hierarchical clustering analysis further highlighted the unique transcriptional profiles induced by bone marrow stromal cell and adipocyte secretomes. From this analysis, gene expression profiles in naive T-cells binned into four clusters when cultured in UCM, SCM, or ACM. Cluster 1 represents genes that were significantly increased in naive T cells cultured in SCM relative to UCM and ACM. Of the top 40 uniquely upregulated genes in cluster 1, notable targets included genes involved in the cellular response to cytokine, actin cytoskeletal regulation, and T-cell activation. Cluster 2 genes were significantly downregulated in ACM compared to UCM and SCM. Among these genes within cluster 2, several transcripts were involved in the response to external biotic stimuli, signal transduction, and cell adhesion. Cluster 3 genes had a similar loss of expression in ACM and SCM compared to UCM. Lastly, cluster 4 represents genes that have an enhanced expression in ACM compared to UCM and SCM. Interestingly, a subset of upregulated cluster 4 genes were found to be involved in cytokine and chemokine activity, nucleosome assembly, apoptosis, and lipid metabolism.

A subset of 13 altered genes of interest were identified as sensitive to ACM or SCM treatment and also involved in TCR signal transduction. Notably, Actb, Lat, Lck, Lcp2, Mapk3, Mapk1, Myh9, Tubg1 were lower in ACM-treated cells compared to UCM or SCM-treated cells. Downregulation of these genes would negatively impact proximal TCR signal transduction (Lat, Lck, Mapk3, Mapk1) and IS formation (Actb, Myh9, Tubg1) 53, which hint at possible mechanisms underlying adipocyte-mediated suppression of T-cell function. Interestingly, increased gene expression levels of Lcp2 and Mapk1, the genes that encode SLP76 and ERK2 respectively, in naive T-cells cultured in SCM were observed. Notably, Lcp2/SLP76 is a master regulator of T-cell function, where it serves as an important adaptor for TCR proximal downstream signaling molecules and a nucleation hub for actin polymerization. An upregulation of Lcp2 in SCM-cultured naive T-cells could potentially explain the enhanced TCR signaling and augmented effector functions observed in T-cells in this condition. In addition, ERK2 phosphorylation occurs soon after TCR triggering and is an important step in the signal transduction cascade that leads to T-cell activation. An upregulation of Mapk1/ERK2 provides an additional explanation for the enhanced TCR signaling. Genes which were upregulated in naive T-cells exposed to the adipocyte secretome include Actg1, Cd69, Cdc42, Nur77, and Zap70. These results indicate that these genes can be induced in an antigen-independent manner and may be upregulated as a compensatory mechanism to overcome adipocyte-mediated T-cell suppression. Indeed, in addition to TCR triggering, Nur77 and CD69 are upregulated in response to fatty acids, prostaglandins, inflammatory cytokines, peptide hormones, cellular stress, T-cell exhaustion, and other physiological stimuli.

### The Soluble Microenvironment Changes Total Actin and Filamentous Actin Levels in OT1 T Cells

Given the number of differentially expressed genes associated with TCR signal transduction, actin nucleation, and the formation of the immunological synapse, experiments were performed to determine whether the bone marrow stromal cell and adipocyte secretomes modulated β-actin and cytoskeletal dynamics in naive T-cells. Differentially expressed genes involved in the regulation of the actin cytoskeleton were identified. Wiskott-Aldrich syndrome protein family member 1 is a protein coded by the Wasf1 (Wave1) gene. A significant increase in the expression of this gene was observed in SCM-cultured T-cells. Wasf1 plays a critical role in the WASP-family verprolin homologous protein (WAVE1) regulatory complex which enhances actin polymerization through its association with Rac and the actin nucleation core Arp2/3 complex. This protein also enhances cytoskeletal membrane ruffling, thereby promoting a motile cell surface. Notably, this was the only actin-related gene that was increased in naive T-cells cultured in SCM relative to other conditions tested. In contrast, ACM promoted genes that inhibit actin cytoskeletal function (Ppp1r12a, Raf1, and Pak1) and downregulated genes that enhance actin polymerization, adhesion, and cytoskeletal activity (Mylk3, Rac2, Itga4, Actn1, Pik3r2, Itga7, Arghef4, Lpar5, Pip4k2b, Pfn2, Iqgap3, Itga1, Pdgfb, and Itga5).

To validate gene expression changes at the protein level in naive T-cells in different microenvironments, total β-actin levels were measured in OT-1 T-cells treated with UCM, SCM, or ACM for 24 hours. A significant decrease in the β-actin/GAPDH ratio was observed in cells treated with ACM compared to SCM with no significant changes in GAPDH levels between conditions. Filamentous actin (F-actin) levels and cell adhesion were measured to determine if these processes were compromised in naive T-cells cultured in ACM as a result of lower levels of β-actin in these cells. OT-1 T-cells were treated for 24 hours with UCM, SCM, or ACM prior to seeding on anti-CD3ε-coated glass for 30 minutes. OT-1 T-cells were then fixed, permeabilized, and stained with SiR-Actin™ to measure F-actin levels via fluorescence microscopy. Compared to UCM, OT1 T cells treated with ACM had a 52% decrease in F-actin levels as well as a 38% decrease in spreading area. In contrast, OT-1 T-cells treated with SCM had a 29% increase in F-actin levels as well as a 14% increase in spreading area.

Additionally, total cholesterol levels were measured after treatment with UCM, SCM, or ACM, because membrane cholesterol can impact membrane stiffness and TCR triggering, and therefore may be responsible for a change in T-cell function. Compared to UCM, a 20% and 24% increase in cholesterol content was observed in ACM and SCM respectively. These results demonstrate that both the bone marrow stromal cell and adipocyte secretomes increase membrane cholesterol levels in naive T-cells, potentially altering membrane fluidity. Even though SCM- and ACM-treated T-cells had similar cholesterol levels, the dysfunctional cytoskeleton in ACM-treated cells will likely make it difficult for these cells to overcome changes in membrane fluidity.

### The Soluble Microenvironment Alters TCR Biomechanics.

Because F-actin polymerization is the primary mediators of TCR forces, experiments were performed to determine the effects of the adipocyte and bone marrow stromal cell secretomes on mechanical forces exerted by the TCR. Forces at pN levels are a component of TCR triggering upon antigen recognition. Since ACM compromised T-cell function and suppressed TCR signal transduction, β-actin protein levels, and F-actin polymerization, it is contemplated that T-cell suppression is mediated by dampened TCR forces upon antigen recognition. Thus, experiments were performed to determine whether SCM treatment enhances TCR biomechanics due to enhanced T-cell signaling upon treatment with SCM and enhanced F-actin levels.

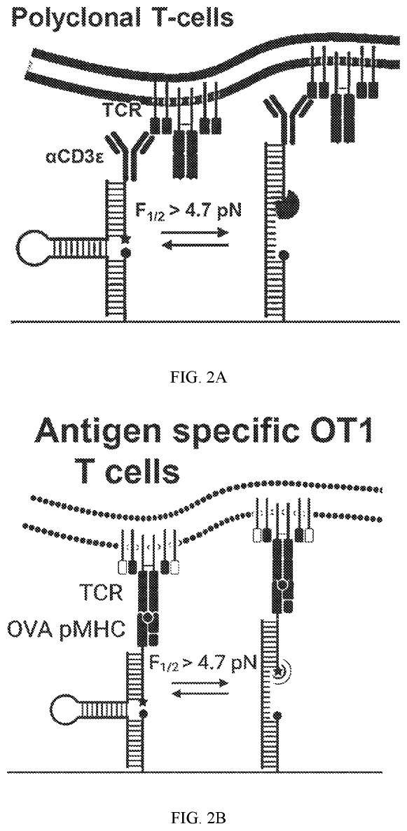

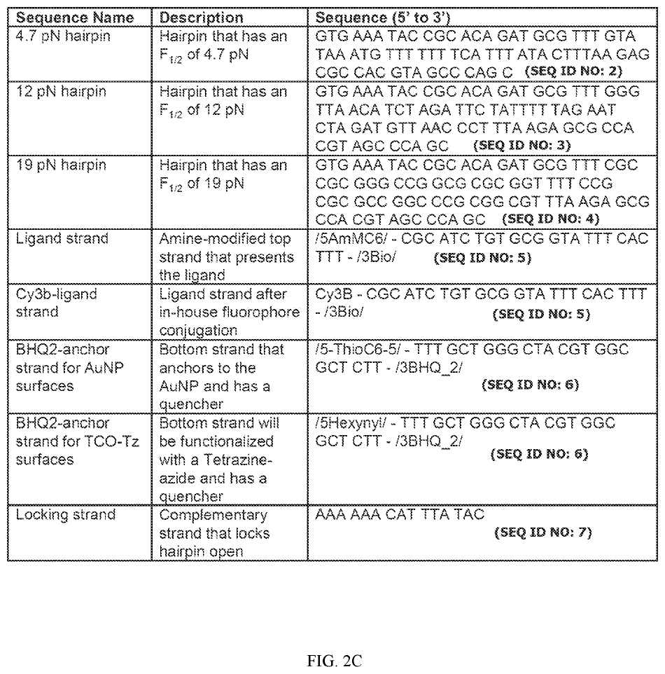

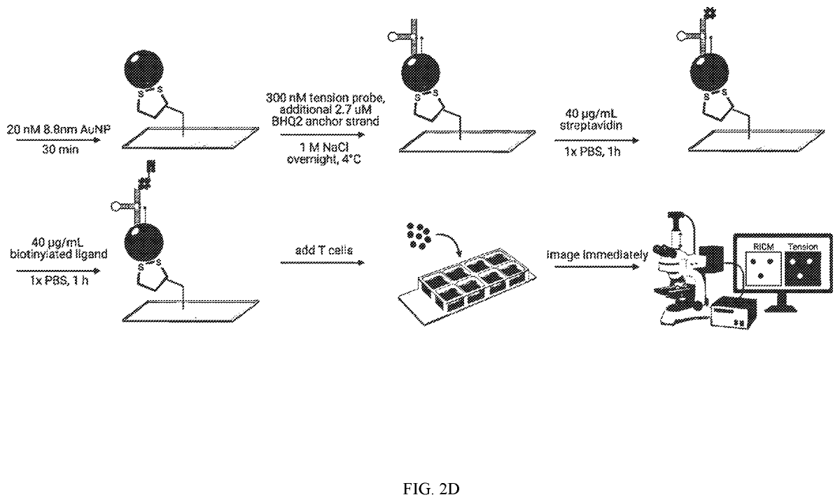

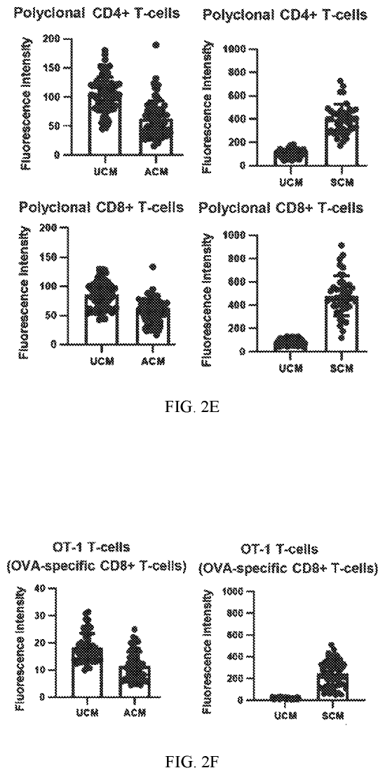

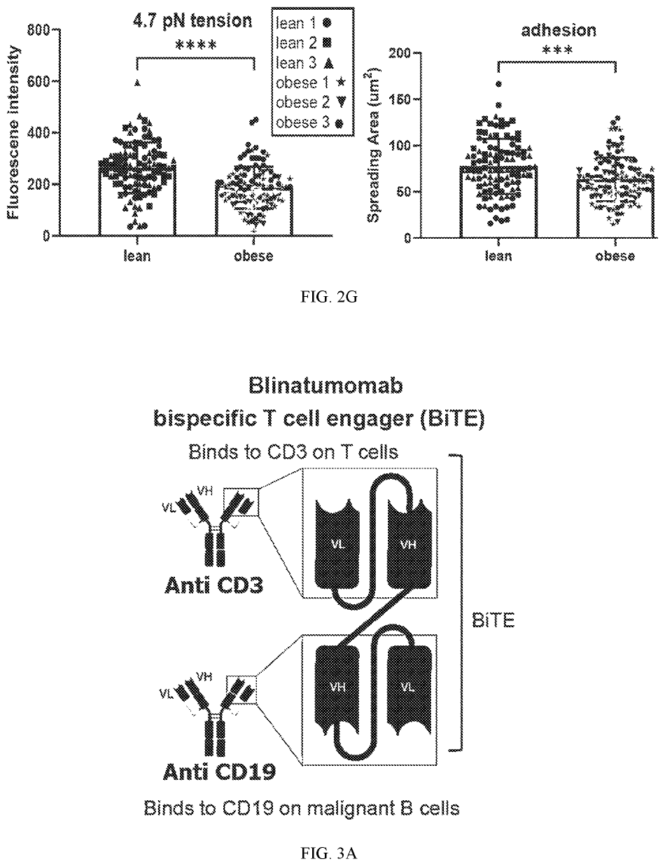

DNA-based tension probes were employed to measure TCR forces during antigen recognition. DNA-based tension probes consist of a DNA duplex that is anchored to a glass surface on one end and presents a ligand of interest to the T-cell on the other end. The DNA duplex has a hairpin that is flanked by a fluorophore-quencher pair, and the hairpin mechanically unfolds when the TCR exerts a threshold mechanical force on the ligand (FIG. 2A). The mechanical unfolding of the hairpin separates the fluorophore and quencher, causing a large increase in fluorescence intensity that can be mapped and measured in real-time by conventional fluorescence microscopy. The force at which the hairpin mechanically unfolds, or the F1/2, is defined by the force at which the hairpin has a 50% probability of unfolding. The Fin is tuned by altering the GC content of the hairpin and the size of the stem loop. In the experiments described herein, a hairpin with F1/2=4.7 pN was used. Isolated polyclonal CD4+ and CD8+ murine T-cells were cultured in UCM, SCM, or ACM for 24 hours before seeding them on DNA-based tension probes presenting an anti-CD3ε antibody as the ligand (FIG. 2A). Relative to UCM, CD4+ and CD8+ T-cells cultured in ACM had a 41% and 34% decrease in 4.7 pN tension signal, respectively. In contrast, CD4+ and CD8+ cultured in SCM had a 278% and 416% increase in 4.7 pN tension signal, respectively.

Next, these responses were validated in antigen-specific T-cells. OT-1 T-cells were cultured in each condition prior to seeding on DNA-based tension probes presenting the H2-K (b) OVA 257-264 SIINFEKL (SEQ ID NO: 1) epitope that is specifically recognized by the OT-1 TCR (FIG. 2B). Relative to UCM, OT1 T-cells cultured in ACM had a 37% decreased in TCR tension signal. In contrast, OT1 T-cells cultured in SCM had a 1240% increase in TCR tension signal. Additionally, OT-1 T-cells treated with SCM were able to exert forces of higher magnitudes (12 and 19 pN) compared to UCM. OT-1 T-cells were unable to mechanically unfold 19 pN hairpin probes unless ICAM was present, demonstrating that the soluble microenvironment can modulate the magnitude of TCR mechanics. Timelapse videos showed that OT-1 TCR forces were highly dynamic for all three environments (UCM, SCM, and ACM), but as quantified above, ACM forces were significantly dampened.

### T-Cell Transmitted Forces to Blinatumomab is Regulated by the Soluble Microenvironment.

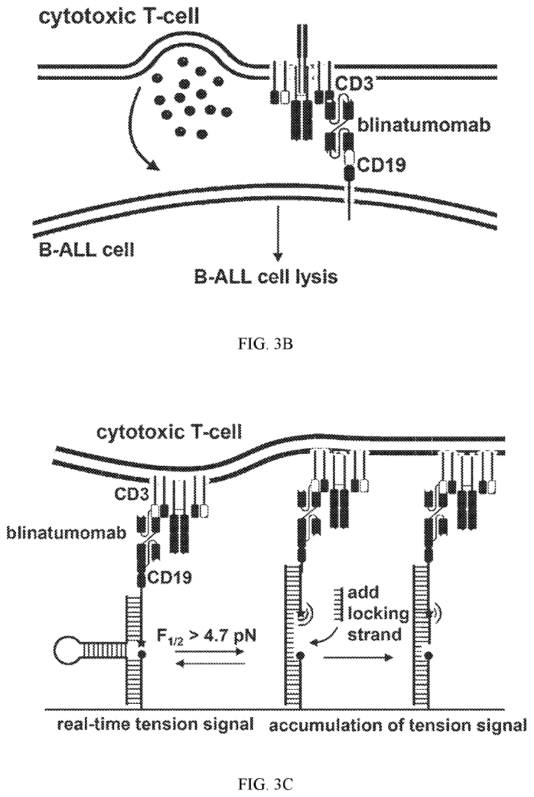

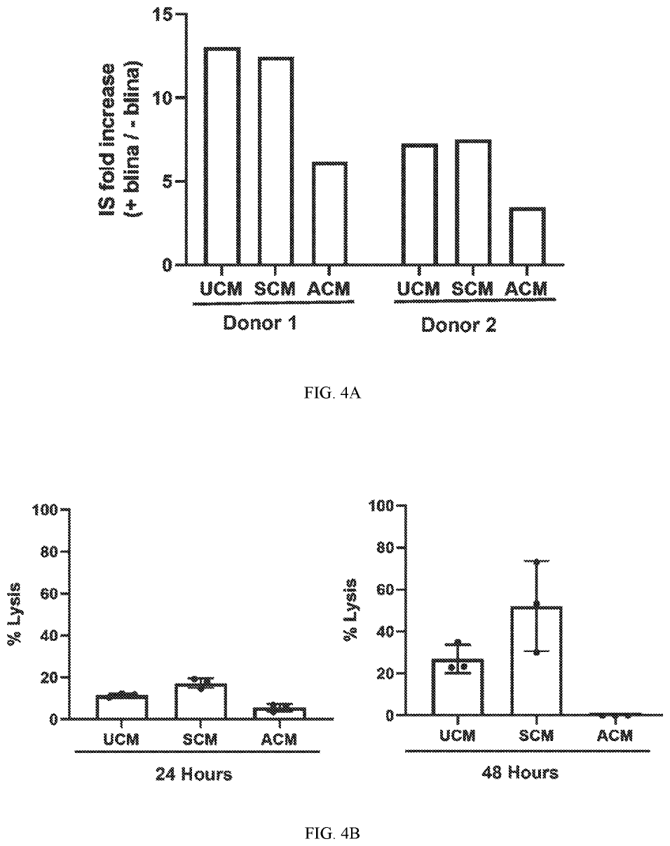

Experiments were performed to determine whether SCM and ACM modulate the magnitude of TCR forces transmitted through the bispecific T-cell engager (BiTE), blinatumomab (FIG. 3A). It is contemplated that SCM and ACM modulation of T-cell function and TCR-antigen forces would be mirrored using BiTEs with human donor T-cells. A DNA-based tension probe that presented CD19 as its ligand was created. Blinatumomab was added to the CD19 probes to mimic the role of the BiTE in patients (FIG. 3B). CD3+ human donor T-cells were seeded to the blinatumomab-presenting probes (“blina-probes”) and the signal was amplified using a DNA strand that is complementary to the stem loop region of the hairpins to lock the probes open after they are mechanically unfolded by the T-cell (FIG. 3C). Using the locking strand, the accumulation of TCR tension signal generated by T-cells cultured in UCM, SCM, or ACM were measured. Compared to UCM, a 24% increase in fluorescent tension signal generated by T-cells exposed to SCM was observed; whereas the ACM resulted in a 53% decrease in fluorescent tension signal when human T-cells were added to blina-probes. To show that binding and tension were specific to blinatumomab, T-cells were seeded on probes presenting only CD19 and no binding or accumulation of fluorescent tension signal was observed. These results demonstrate that human TCR biomechanical forces are modulated by the soluble microenvironment and dictate the strength of the interaction with blinatumomab.

The soluble microenvironment impacts immunological synapse formation and blinatumomab-mediated killing of malignant B-cells. Since mechanical forces are important to TCR triggering, immunological synapse formation, and cytotoxicity, experiments were designed to determine if reduced mechanical forces through blinatumomab correlated with reduced T cell function. Immunological synapse formation between human CD3+ T-cells and human B-ALL cells was assessed using ImageStream™ flow cytometry. The fold increase in immunological synapse formation was measured in human donor T-cells cells treated with blinatumomab compared to a vehicle control, and immunological synapse formation was determined using the distance between the T-cell and target cell and the actin brightness within the cell-cell interface. Using human T-cells from two donors, similar levels of immunological synapse formation was observed between UCM and SCM treated cells in the presence of blinatumomab, whereas ACM reduced blinatumomab-induced immunological synapse formation. Human T-cell-mediated cytolysis of human B-ALL cells was assessed in the absence and presence of blinatumomab after 24 and 48 hours of co-culture. Compared to UCM, human T-cells exposed to the bone marrow stromal cell secretome had a 51% increase in human B-ALL cell lysis while T-exposed to the adipocyte secretome exhibited a 51% decrease in human B-ALL cell lysis. After 48 hours of co-culture, increased killing was observed for human T-cells cultured in UCM and SCM; whereas, human T-cells failed to kill target B-ALL cells after 2 days of co-culture with blinatumomab in the adipocyte secretome.

### Gold Nanoparticle Surface Preparation for Murine T-Cell Probes

Gold nanoparticle DNA probe surfaces were prepared on No. 2 glass coverslips were placed in a rack and immersed and sonicated in water for 10 minutes followed by sonication in 200 proof ethanol for 10 minutes. After sonication, the coverslips were baked in an 80° C. oven to evaporate all remaining ethanol. Once completely dry, the rack containing coverslips was immersed in 40 mL freshly made piranha solution (3:1 v/v mixture of H₂SO₄:H₂O₂) for 30 minutes. After 30 minutes, the slides were washed with 40 ml water 6 times to remove all piranha solution. Slides were then washed 3 times with 200 proof ethanol to remove all water. Slides were then incubated in 3% APTES in 50 mL 200 proof ethanol for 1 hour at room temperature. After incubation, slides were washed 3 times with 200 proof ethanol and baked in an 80° C. oven for 30 minutes. The amine-modified coverslips were allowed to cool for 5 minutes and placed in petri dishes lined with parafilm. 200 μL of 0.5% w/v lipoic acid-PEG-NHS and 2.5% w/v mPEG-NHS in 0.1M NaHCO₃ was added to each coverslip was incubated for 1 h at room temperature. Coverslips were rinsed with water and incubated with 1 mg/mL sulfo-NHS-acetate in 0.1M NaHCO₃ for 30 minutes to passivate any unreacted amine groups. Coverslips were intensively washed with water and then incubated with 500 μL of 20 nM gold nanoparticles (8.8 nm, tannic acid modified) for 30 minutes at room temperature. Meanwhile, DNA hairpin probes were assembled in 1M NaCl by mixing the 4.7 pN hairpin strand (0.3 μM), Cy3b-ligand strand (0.3 μM), and BHQ2-anchor strand (0.3 μM) at a ratio of 1:1.1:1.1. The mixture was annealed by heating to 95° C. for 5 minutes and cooling at 25° C. for 30 minutes. After annealing, an additional 2.7 μM of the BHQ2 anchor strand was added to the DNA solution for additional quenching. After the 30 minute incubation with gold nanoparticles, coverslips are rinsed extensively with water, followed by rinsing with 1 M NaCl. Next, the annealed DNA probe and quencher solution was added to the coverslips and incubated overnight in the dark at 4° C. The next day, coverslips were washed with PBS and 40 μg/mL of streptavidin in PBS was added to the functionalized surface for 1 h at room temperature. The coverslips were washed with PBS, then 40 μg/mL of biotinylated αCD3ε or OVA-pMHC was added to the functionalized surface for 1 h at room temperature. Finally, the slides were washed with PBS, assembled in imaging chambers, and immediately used for imaging.

### TCO Surface Preparation for Blinatumomab Tension Probes.

TCO-glass surfaces were prepared identical to the AuNP coverslips through the APTES step. 100 μL of 4 mg/mL TCO-NHS in DMSO was added to each amine-functionalized coverslip and another coverslip was placed on top to create a “sandwich.” Coverslips were incubated for at least 12 h or up to 1 week at RT. Coverslips were intensively washed with ethanol then PBS to remove all DMSO, and then incubated with 0.1% BSA in PBS for 30 min to block any nonspecific interactions. Meanwhile, DNA hairpin probes were assembled in 1M NaCl by mixing the 4.7pN hairpin strand (0.2 μM), Cy3b-ligand strand (0.22 μM), and BHQ2-anchor strand (0.22 μM) at a ratio of 1:1.1:1.1. The mixture was annealed by heating to 95° C. for 5 minutes and cooling at 25° C. for 30 minutes. After the 30 minute incubation with 0.1% B BSA, coverslips are rinsed extensively with PBS. Next, the annealed DNA probe solution was added to the coverslips and incubated for 1 h at RT. Next, coverslips were washed with PBS and 40 μg/mL of streptavidin in PBS was added to the functionalized surface for 1 h at room temperature. The coverslips were washed with PBS, then 10 μg/mL of biotinylated CD19 protein was added to the functionalized surface for 1 h at room temperature. Next, 10 μg/mL blinatumomab in PBS was added to CD19 probes for 1 h. Finally, the slides were washed with PBS, assembled in imaging chambers, and immediately used for imaging.

Labeling DNA Ligand Strand with Cy3B Via NHS Coupling

Oligonucleotides were synthesized to include a terminal amine for NHS-Cy3b coupling. Briefly, an excess of Cy3b-NHS dye (50 μg) was dissolved in DMSO and added to a solution containing 10 nmol of the DNA ligand strand in 1×PBS with 0.1M NaHCO₃. The reaction incubated at room temperature overnight. The next day, byproducts, salts, and unreacted Cy3B-NHS was removed via gel filtration in a centrifugal device. The DNA-Cy3B conjugate was further purified by reverse-phase HPLC.

Labeling DNA Anchor Strand with Tetrazine Using a Copper Click Reaction

Tetrazine labeling of oligonucleotides were synthesized to include a BHQ2 quencher and a terminal alkyne for azide-tetrazine coupling. Alkyne-modified DNA was reacted with an excess amount of methyltetrazine-PEG4-Azide in the presence of CuSO₄ (0.20 μmol), sodium ascorbate (0.50 μmol), and THPTA (0.25 μmol) in 40 μL (1:1 ratio of DMSO:water) for 1 hour at 50° C. The product was then filtered using gel filtration in a centrifugal device. The tetrazine-DNA conjugate was further purified by reverse-phase HPLC.

What is claimed is:

1. A method of detecting a light and/or fluorescent signal from a blood sample of a subject comprising a cell expressing a CD3 antigen comprising the steps of:
a) exposing a device to a cell comprising CD3 antigen and a bi-specific T-cell engager that specifically binds a CD3 antigen and a second antigen in the presence of a locking oligonucleotide;
wherein the device comprises:
i) a second antigen;
ii) a nucleic acid complex linker having a first end and a second end, wherein the nucleic acid complex linker is linked to the second antigen at the first end;
iii) a surface connected to the nucleic acid complex linker at the second end;
iv) a quencher conjugated to the nucleic acid complex linker; and
v) a first fluorescent molecule conjugated to the nucleic acid complex linker wherein the fluorescent molecule is in a position and configured to move the position away from quencher when the second antigen moves away from the quencher;
wherein the nucleic acid complex linker comprises a hairpin motif comprising a double stranded stem segment, a single stranded loop segment, a first end tail segment, and a second end tail segment;
wherein the quencher and the first fluorescent molecule are configured to quench when the nucleic acid complex linker is in the form of a hairpin motif;
wherein the quencher and the first fluorescent molecule are not configured to quench when the nucleic acid complex linker is in the form of a single stranded motif; and
wherein the bi-specific T-cell engager binds CD3 and the second antigen and pulls the second antigen away from the surface to unravel the hairpin motif into the single stranded motif removing the first fluorescent molecule from proximity to the quencher producing a light and/or fluorescent signal and the locking oligonucleotide hybridizes to the single stranded motif under conditions such that the nucleic acid complex linker is locked in an extended form; and
b) detecting the light and/or fluorescent signal.

2. The method of claim 1 wherein the subject is receiving a chemotherapy treatment including a bi-specific T-cell engager that specifically binds both CD3 and the second antigen.

3. The method of claim 1 wherein the bi-specific T-cell engager is blinatumomab.

4. The method of claim 2 wherein the detected light and/or fluorescent signal indicates an increased risk that the subject may not be responsive to the chemotherapy treatment or that the chemotherapy treatment has stopped working.

5. The method of claim 2 wherein the detected light and/or fluorescent signal indicates an increased risk that the subject is experiencing cytokine release syndrome.

6. The method of claim 1 wherein the sample is from subject that has not yet received a chemotherapy treatment including a bi-specific T-cell engager, then is to be considered for such treatment.

7. The method of claim 1, wherein the locking oligonucleotide comprises a sequence that is only one nucleotide that base pairs with the last nucleotide of the double stranded stem segment followed by the reverse complement of the single stranded loop segment followed by the reverse complement of the double stranded stem segment.

8. The method of claim 1, wherein the second antigen is cluster of differentiation 19 (CD19), cluster of differentiation 10 (CD10), cluster of differentiation 20 (CD20), cluster of differentiation 33 (CD33), cluster of differentiation 38 (CD38), CD70 (tumor necrosis factor ligand superfamily member 7), CD133 (prominin 1), CD171 (L1 cell adhesion molecule), (EGFR) epidermal growth factor receptor, (HER2) human epidermal growth factor receptor 2, EGFR vIII (epidermal growth factor receptor variant 3) (MUC1) mucin1, (MUC16) mucin16, (EpCAM) epithelial cell adhesion molecule, (AFP) alpha-fetoprotein, (FAP) familial adenomatous polyposis, (CEA) carcinoembryonic antigen, (PSCA) prostate stem cell antigen, (PSMA) prostate-specific membrane antigen, (PSA) prostate-specific antigen, (AXL) AXL receptor tyrosine kinase, (DLL3) delta-like 3, (EPHA2) EPH receptor A2, (FRα) folate receptor alpha, (LMP1) Epstein-Barr virus latent membrane protein 1, (MAGE) melanoma antigen gene protein, MAGE-A1, MAGE-A3, MAGE-A4, (DR5) death receptor 5, (NKG2D) natural killer group 2 member D receptor, (CAIX) carbonic anhydrase IX, (TAG-72) tumor-associated glycoprotein 72, (GUCY2C) guanylate cyclase 2C, (ANTXR1) anthrax toxin receptor 1, (GSPG4) general secretion pathway protein G, (ROR) RAR-related orphan receptors, ROR1 (receptor tyrosine kinase like orphan receptor 1), IL13RA2 (Interleukin 13 Receptor Subunit Alpha 2), Wilms' tumor 1 (WT1), Survivin, Tn (aGalNAc-O-Ser/Thr), sialyl-Tn (aNeuAc2,6-aGalNAc-O-Ser/Thr), TF (bGal1,3-aGalNAc-O-Ser/Thr), CA 19-9 (Neu5Acα2-3Galβ1-3 [Fucα1-4]GlcNAcβ), Telomerase reverse transcriptase (TERT), Beta-hCG (Human chorionic gonadotropin), p53, Ras, bladder tumor antigen (BTA), antibody specific antigen Om5, GD2 (Ganglioside GD2), integrin alpha-v/beta-6, or mesothelin antigen, BCMA (TNF receptor superfamily member 17\B-cell maturation protein), CD123 (interleukin 3 receptor subunit α\CD123 antigen), CD138 (syndecan 1), CD22 (SIGLEC2), CD5 (lymphocyte antigen T1/Leu-1), Ig kappa chain, LeY (fucosyltransferase 3/Lewis Blood Group), NKG2D ligand (killer cell lectin like receptor K1/CD314), WT1 (Wilms' tumor antigen 1), C-Met (MET proto-oncogene), CAIX (carbonic anhydrase 9), GPC3 (glypican 3), HPV16-E6 (human papillomavirus E6 protein), MARTI (melan-A), NY-ESO-1 (cancer/testis antigen 1B), PD-L1 (CD274 molecule), PSMA (folate hydrolase 1), or VEGFR2 (kinase insert domain receptor/vascular endothelial growth factor receptor 2).

9. A device comprising:
i) an antigen found on cancer cells;
ii) a nucleic acid complex linker having a first end and a second end, wherein the nucleic acid complex linker is linked to the antigen at the first end;
iii) a surface connected to the nucleic acid complex linker at the second end;
iv) a quencher conjugated to the nucleic acid complex linker; and
v) a first fluorescent molecule conjugated to the nucleic acid complex linker, wherein the fluorescent molecule is in a position and configured to move the position of the fluorescent molecule away from the quencher relative to the quencher when the antigen moves upon binding to a receptor of the antigen;
wherein the nucleic acid complex linker comprises a hairpin motif comprising a double stranded stem segment, a single stranded loop segment, a first end tail segment, and a second end tail segment;
wherein the quencher and the first fluorescent molecule are configured to quench when the nucleic acid complex linker is in the form of a hairpin motif; and
wherein the quencher and the first fluorescent molecule are not configured to quench when the nucleic acid complex linker is in the form of a single stranded motif.

10. The device of claim 9 wherein the antigen is cluster of differentiation 19 (CD19), cluster of differentiation 10 (CD10), cluster of differentiation 20 (CD20), cluster of differentiation 33 (CD33), cluster of differentiation 38 (CD38), CD70 (tumor necrosis factor ligand superfamily member 7), CD133 (prominin 1), CD171 (L1 cell adhesion molecule), (EGFR) epidermal growth factor receptor, (HER2) human epidermal growth factor receptor 2, EGFR vIII (epidermal growth factor receptor variant 3) (MUC1) mucin1, (MUC16) mucin16, (EpCAM) epithelial cell adhesion molecule, (AFP) alpha-fetoprotein, (FAP) familial adenomatous polyposis, (CEA) carcinoembryonic antigen, (PSCA) prostate stem cell antigen, (PSMA) prostate-specific membrane antigen, (PSA) prostate-specific antigen, (AXL) AXL receptor tyrosine kinase, (DLL3) delta-like 3, (EPHA2) EPH receptor A2, (FRα) folate receptor alpha, (LMP1) Epstein-Barr virus latent membrane protein 1, (MAGE) melanoma antigen gene protein, MAGE-A1, MAGE-A3, MAGE-A4, (DR5) death receptor 5, (NKG2D) natural killer group 2 member D receptor, (CAIX) carbonic anhydrase IX, (TAG-72) tumor-associated glycoprotein 72, (GUCY2C) guanylate cyclase 2C, (ANTXR1) anthrax toxin receptor 1, (GSPG4) general secretion pathway protein G, (ROR) RAR-related orphan receptors, ROR1 (receptor tyrosine kinase like orphan receptor 1), IL13RA2 (Interleukin 13 Receptor Subunit Alpha 2), Wilms' tumor 1 (WT1), Survivin, Tn (aGalNAc-O-Ser/Thr), sialyl-Tn (aNeuAc2,6-aGalNAc-O-Ser/Thr), TF (bGal1,3-aGalNAc-O-Ser/Thr), CA 19-9 (Neu5Acα2-3Galβ1-3 [Fucα1-4]GlcNAcβ), Telomerase reverse transcriptase (TERT), Beta-hCG (Human chorionic gonadotropin), p53, Ras, bladder tumor antigen (BTA), antibody specific antigen Om5, GD2 (Ganglioside GD2), integrin alpha-v/beta-6, or mesothelin antigen, BCMA (TNF receptor superfamily member 17\B-cell maturation protein), CD123 (interleukin 3 receptor subunit α\CD123 antigen), CD138 (syndecan 1), CD22 (SIGLEC2), CD5 (lymphocyte antigen T1/Leu-1), Ig kappa chain, LeY (fucosyltransferase 3/Lewis Blood Group), NKG2D ligand (killer cell lectin like receptor K1/CD314), WT1 (Wilms' tumor antigen 1), C-Met (MET proto-oncogene), CAIX (carbonic anhydrase 9), GPC3 (glypican 3), HPV16-E6 (human
